(19) United States
(12) Patent Application Publication
(10) Pub. No.: US 2006/0127964 A1
(43) Pub. Date: Jun. 15, 2006

(54) Microprocessors, devices, and methods for use in monitoring of physiological analytes

(76) Inventors: Russell Ford, Watsonville, CA (US); Matthew J. Lesho, Elicott City, MD (US); Janet A. Tamada, Stanford, CA (US); Michael J. Tierney, San Jose, CA (US)

Correspondence Address: RATNERPRESTIA, P O BOX 980, VALLEY FORGE, PA, 19482-0980, US

(21) Appl. No.: 11/350,193

(22) Filed: Feb. 8, 2006

Related U.S. Application Data
(62) Division of application No. 10/903,672, filed on Jul. 30, 2004.

(51) Int. Cl. C12Q 1/54; G06F 19/00

(52) U.S. Cl. 435/14; 702/19

(57) ABSTRACT

Described herein are microprocessors, devices, and methods useful for sweat and/or temperature detection that correlate more closely with changes in amperometric or charge signals related to analyte amount or concentration. The present invention provides methods for the establishment of more accurate sweat and/or temperature thresholds and new methods of compensation, such as correcting for the effects of sweat and rapidly changing temperature on measured analyte values. The present invention reduces the number of skipped or unuseable readings provided by analyte monitoring devices during periods of sweating or changing temperatures. Further, the present invention provides methods for improving the accuracy of reported readings of analyte amount or concentration. In one aspect, the present invention provides passive collection reservoir/sensing devices used in combination with active collection reservoir/sensing devices for detection of sweat and/or temperature related parameters.

### CROSS-REFERENCE TO RELATED APPLICATIONS

This application is a Divisional application of U.S. application Ser. No. 10/903,672, filed Jul. 30, 2004 and claims the benefit of U.S. Provisional Application No. 60/495,294, filed Aug. 15, 2003, which applications are herein incorporated by reference in their entirety.

### TECHNICAL FIELD

The present invention relates generally to microprocessors, devices, and methods of monitoring of physiological analytes and detection of amounts or concentrations of such analytes. In one aspect, the present invention relates to improved selectivity of data screens. In another aspect, the present invention relates to compensation for fluctuations (e.g., sweat and/or temperature) that affect analyte measurement.

### BACKGROUND OF THE INVENTION

The transdermal migration of numerous biological substances are known to be affected by sweating. For example, in studying transcutaneous chemical collection devices and the phenomenon of outward transcutaneous chemical migration, it was observed that sweating seemed to have a large (40%) contribution to transdermal collection in an early collection period (5.5 hours) with a reduction of the difference (14%) at longer collection times (10 hours) (Conner, D. P., et al., J. Invest. Dermatol. 96(2): 186-90, 1991). Some substances of interest can be detected in sweat samples, for example, cocaine and codeine (Huestis, M. A., et al., J. Chromatogr. B. Biomed. Sci. Appl. 15;733(1-2):247-64, 1999), caffeine, paraxanthine, and theobromine (Delahunty, T., et al., J. Anal. Toxicol. 22(7):596-600, 1998), chloride (for example, in the diagnosis of cystic fibrosis, Kabra, S. K., et al., Indian Pediatr. 39(11):1039-43, 2002), potassium (Lande, G., Int. J. Cardiol. 77(2-3):323-4, 2001), amino acids (Cynober, L. A., Nutrition 18(9):761-6, 2002), chromium (Davies, S., et al., Metabolism 46(5):469-73, 1997), electrolytes, glucose (Tamada, J. A., et al., JAMA 282(19):1839, 1999) and urea (al-Tamer, Y. Y., et al., Eur. J. Clin. Chem. Clin. Biochem. 32(2):71-7, 1994).

Analyte levels determined through use of transdermal analyte monitoring devices and/or the function of the monitoring devices may also be affected by sweating. For example, the integrated current related to glucose concentration, as measured by the GlucoWatch® (Cygnus, Inc., Redwood City, Calif.) biographer systems, can be affected by sweat (see, e.g., GlucoWatch G2® (Cygnus, Inc., Redwood City, Calif.) Automated Glucose Biographer product insert sheet). To maintain accuracy in the measured glucose value, the GlucoWatch biographer systems account for the effects of sweat by using sweat probes that measure changes in skin conductivity. When the skin conductivity exceeds a pre-selected threshold value, associated readings from the GlucoWatch biographer systems are skipped (see, e.g., GlucoWatch G2 Automated Glucose Biographer User's Guide). Rapid temperature changes may also cause the GlucoWatch biographer systems to skip a reading.

Generally, transdermal analyte monitoring systems must address problems associated with sweat and temperature changes. Minimally invasive analyte (e.g., glucose) monitoring methods, for example, such as those using microneedles, microporation (e.g., by laser or thermal ablation), sonophoresis, suction, skin permeabilization, are all affected by analyte collected via perspiration versus analyte collected by the sampling method. An RF-impedance device that measures glucose under the skin has been described (Caduff, A., et al., American Diabetes Association 62^{nd} Scientific Sessions, San Francisco, Jun. 14-18, 2002, Diabetes 51:(Supp.2), A119, 2002). Perspiration may provide interference in such a device that measures glucose under the skin via RF-impedance. Accordingly, transdermal spectroscopic methods may also be affected by an extra glucose on the surface of the skin in sweat.

Current methods of sweat and temperature detection are typically only loosely correlated with changes in amperometric or charge signals. Therefore, tight thresholds are usually set with regard to sweat and temperature change to avoid degraded accuracy in the resulting glucose readings.

The microprocessors, systems, and methods of the present invention provide improved temperature and sweat detection that correlate more closely with changes in amperometric or charge signals. Further, the present invention provides for the establishment of more accurate thresholds and more accurate compensation for the effects of sweat and/or rapidly changing temperature, both of which result in improved accuracy of analyte monitoring devices.

### SUMMARY OF THE INVENTION

The present invention relates to microprocessors, devices, and methods of monitoring of physiological analytes and detection of amounts or concentrations of such analytes.

In one aspect, the present invention relates to one or more microprocessors comprising programming to control performance of the following steps. The one or more microprocessors provide a first signal related to analyte amount or concentration in a subject from a first sample comprising an analyte, wherein the first sample is obtained by use of a method that enhances transport of the analyte across a skin or mucosal surface of the subject. Further, the one or more microprocessors provide a second signal related to analyte amount or concentration from a second sample comprising the analyte, wherein the second sample is obtained substantially without use of a method that enhances transport of the analyte across the skin or mucosal surface of the subject, and the first signal and the second signal are obtained for substantially a same time period. The one or more microprocessors then qualify the first signal, for example, by a method selected from the group consisting of (i) screening the first signal based on the second signal; (ii) applying a correction algorithm to the first signal, wherein the first signal is adjusted by use of the second signal; and (iii) combinations thereof.

In one embodiment, the qualifying comprises screening the first signal based on the second signal. For example, the screening comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, the screening may compare a signal trend to a predetermined set of signal trends, and the skipping or accepting may be based on matches between the signal trend and one or more predetermined set of signal trends.

In another embodiment, the qualifying further comprises obtaining a skin conductance value for substantially the same time period as the first and second signals, comparing the skin conductance value to a predetermined skin conductance threshold value, and if the skin conductance value equals or exceeds the skin conductance threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high signal threshold value and the low signal threshold value. Alternatively or in addition, a trend of skin conductance values may be compared to a set of predetermined trends of skin conductance values and a decision to further screen the signal may be based on matches between the skin conductance trend and one or more predetermined set of skin conductance trends. Further, subsequent screening may compare a signal trend to a predetermined set of signal trends, and the skipping or accepting may be based on matches between the signal trend and one or more predetermined set of signal trends.

In yet another embodiment, the qualifying further comprises obtaining a temperature value for substantially the same time period as the first and second signals, comparing the temperature value to a predetermined high and/or low temperature threshold value, and if the temperature value is above the high temperature threshold value or below the low temperature threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, a trend of temperature values may be compared to a set of predetermined trends of temperature values and a decision to further screen the signal may be based on matches between the temperature trend and one or more predetermined set of temperature trends. Further, subsequent screening may compare a signal trend to a predetermined set of signal trends, and the skipping or accepting may be based on matches between the signal trend and one or more predetermined set of signal trends.

In additional embodiments, the qualifying comprises use of both of the above-described analyses for skin temperature values (or trends) and temperature values (or trends) before applying further screens.

In a further embodiment, after accepting the first signal for determination of an associated analyte measurement value a correction algorithm is applied to the first signal, for example, by adjusting the first signal using the second signal. In an exemplary adjustment, the correction algorithm comprises correcting the first signal by subtracting at least a portion of the second signal. For example, when the first and second signal are amperometric or coulometric, the correction algorithm comprises Q=Qₐ−kQₚ, where Q is a signal input for determination of an analyte measurement value, Qₐ is the first signal, k is a proportionality factor that is a value between 0 and 1 (and may include the values 0 or 1), and Qₚ is the second signal. As a further example, a correction algorithm comprises correcting the first signal by subtracting at least a portion of the second signal, further taking into account the second signal at a calibration time point. One such correction algorithm comprises Q=Qₐ−k(Qₚ−Q_{pcal}) where Q is a signal input for determination of an analyte measurement value, Qₐ is the first signal, k is a proportionality factor that is a value between 0 and 1 (and may include the values 0 or 1), Qₚ is the second signal, and Q_{pcal} is the second signal at the calibration time point.

Exemplary methods to enhances transport of the analyte across a skin or mucosal surface of the subject include, but are not limited to, iontophoresis, sonophoresis, suction, electroporation, thermal poration, laser poration, use of microporation, use of microneedles, use of microfine lances, skin permeabilization, chemical permeation enhancers, use of laser devices, and combinations thereof. In preferred embodiments iontophoresis, sonophoresis, or laser poration are used.

Exemplary signals that may be employed in the practice of the present invention include, but are not limited to, electrical and chemical signals. In one embodiment, the signal is an electrochemical signal combining conversion of an analyte to a detectable species (such as hydrogen peroxide) and electrical detection of the detectable species (for example, by reaction of hydrogen peroxide at a reactive surface of a sensing electrode). Such an electrochemical signal may be, for example, amperometric or coulometric signal. In one embodiment, the analyte is glucose and the electrochemical signal is obtained by contacting glucose with glucose oxidase and a sensing electrode.

Analytes that can be measured using the microprocessors, methods and devices of the present invention include, but are not limited to, amino acids, enzyme substrates or products indicating a disease state or condition, other markers of disease states or conditions, drugs of abuse (e.g., ethanol, cocaine), therapeutic and/or pharmacological agents (e.g., theophylline, anti-HIV drugs, lithium, anti-epileptic drugs, cyclosporin, chemotherapeutics), electrolytes, physiological analytes of interest (e.g., urate/uric acid, carbonate, calcium, potassium, sodium, chloride, bicarbonate (CO₂), glucose, urea (blood urea nitrogen), lactate and/or lactic acid, hydroxybutyrate, cholesterol, triglycerides, creatine, creatinine, insulin, hematocrit, and hemoglobin), blood gases (carbon dioxide, oxygen, pH), lipids, heavy metals (e.g., lead, copper), and the like. In a preferred embodiment, the analyte is glucose.

The one or more microprocessors of the present invention, in some embodiments, further comprise programming to control operating a first sensing device that provides the first signal and operating a second sensing device that provides the second signal. Further, in some embodiments, the one or more microprocessors of the present invention comprise programming to control operating a first sampling device (e.g., employing an iontophoretic method) that provides the first sample.

The present invention also includes analyte monitoring devices that comprise the one or more microprocessors described herein. Such analyte monitoring devices may, for example, comprise one or more microprocessors and first and second electrochemical sensing devices. Further, such analyte monitoring devices may, for example, comprise one or more microprocessors, first and second electrochemical sensing devices, and a sampling device (e.g., where the sampling device employs iontophoresis, sonophoresis, or microporation, for example, using a laser).

In -one aspect, the present invention relates to an analyte monitoring device comprising, (A) one or more collection reservoirs adapted for contact with a skin or mucosal surface of a subject, wherein (i) movement of the analyte into the collection reservoirs is enhanced by a transdermal or transmucosal sampling method, and (ii) during use of the device at least one collection device is placed in operative contact with an analyte sensing device; and (B) one or more collection reservoirs adapted for contact with a skin or mucosal surface of a subject, wherein (i) movement of the analyte into the collection reservoirs not enhanced by the transdermal or transmucosal sampling method, and (ii) during use of the device at least one collection device is placed in operative contact with an analyte sensing device. In one embodiment, during use of the device at least one collection reservoir of (B) is in contact with a thermistor.

In a preferred embodiment, the physical characteristics of at least one collection reservoir of (A) are substantially the same as the physical characteristics of at least one collection reservoir of (B). An exemplary collection reservoir is a hydrogel.

The analyte monitoring device, in some embodiments, comprises an analyte sensing device that detects the analyte electrochemically. Such a device typically comprises a sensing electrode. In a preferred embodiment, the physical characteristics of the sensing electrode in contact with at least one collection reservoir of (A) has substantially the same physical characteristics of the sensing electrode in contact with at least one collection reservoir of (B). Further, in some embodiments, the analyte sensing device comprises an enzyme to facilitate electrochemical detection of the analyte (e.g., when the analyte is glucose and the enzyme comprises glucose oxidase).

In one embodiment, the analyte monitoring device further comprises iontophoretic electrodes in contact with the one or more collection reservoirs of (A). The device may also comprise iontophoretic electrodes in contact with the one or more collection reservoirs of (B) but, in such instance, the iontophoretic electrodes are typically not connectable to the iontophoretic circuit, that is the iontophoretic electrodes are not activatable to use for extraction.

In yet another embodiment, a collection reservoir of (B) of the analyte monitoring device comprises first and second surfaces, the first surface is in contact with a sensing device and the second surface is in contact with a membrane substantially impermeable to analyte, and the membrane is adapted for contact with the skin or mucosal surface.

In another aspect the present invention comprises a method of qualifying a signal related to an analyte amount or concentration in samples obtained by use of a method that enhances transport of the analyte across a skin or mucosal surface of a subject (e.g., a human). The method typically comprises providing a first signal related to analyte amount or concentration in the subject from a first sample comprising the analyte, wherein the first sample is obtained by use of a method that enhances transport of the analyte across a skin or mucosal surface of the subject. In addition a second signal is provided related to analyte amount or concentration from a second sample comprising the analyte, wherein the second sample is obtained substantially without use of a method that enhances transport of the analyte across the skin or mucosal surface of the subject, and the first signal and the second signal are obtained for substantially a same time period. The first signal may be qualified by a method, for example, selected from the group consisting of (i) screening the first signal based on the second signal; (ii) applying a correction algorithm to the first signal, wherein the first signal is adjusted by use of the second signal; and (iii) combinations thereof.

In one embodiment of the method, the qualifying comprises screening the first signal based on the second signal. For example, the screening comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, the screening may compare a signal trend to a predetermined set of signal trends, and the skipping or accepting may be based on matches between the signal trend and one or more predetermined set of signal trends.

In another embodiment of the method of the present invention, the qualifying further comprises obtaining a skin conductance value for substantially the same time period as the first and second signals, comparing the skin conductance value to a predetermined skin conductance threshold value and, if the skin conductance value equals or exceeds the skin conductance threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, a trend of skin conductance value may be compared to a set of predetermined trends of skin conductance values and a decision to further screen the signal may be based on matches between the skin conductance trend and one or more predetermined set of skin conductance trends. Further, subsequent screening may compare a signal trend to a predetermined set of signal trends, and the skipping or accepting may be based on matches between the signal trend and one or more predetermined set of signal trends.

In a further embodiment of the method, the qualifying further comprises obtaining a temperature value for substantially the same time period as the first and second signals, comparing the temperature value to a predetermined high and/or low temperature threshold value, and if the temperature value is above the high temperature threshold value or below the low temperature threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high signal threshold value and the low signal threshold value. Alternatively or in addition, a trend of temperature values may be compared to a set of predetermined trends of temperature values and a decision to further screen the signal may be based on matches between the temperature trend and one or more predetermined set of temperature trends. Further, subsequent screening may compare a signal trend to a predetermined set of signal trends, and the skipping or accepting may be based on matches between the signal trend and one or more predetermined set of signal trends.

In a further embodiment of the present method, after accepting the first signal for determination of an associated analyte measurement value a correction algorithm is applied to the first signal, for example, by adjusting the first signal using the second signal. In an exemplary adjustment, the correction algorithm comprises correcting the first signal by subtracting at least a portion of the second signal. For example, in some embodiments when the first and second signals are amperometric or coulometric, the correction algorithm comprises Q=Qₐ−kQₚ, where Q is a signal input for determination of an analyte measurement value, Qₐ is the first signal, k is a proportionality factor that is a value between 0 and 1 (and may include the values 0 or 1), and Qₚ is the second signal. As a further example, a correction algorithm in some embodiments comprises correcting the first signal by subtracting at least a portion of the second signal, further taking into account the second signal at a calibration time point. One exemplary correction algorithm comprises Q=Qₐ−k(Qₚ−Q_{pcal}) where Q is a signal input for determination of an analyte measurement value, Qₐ is the first signal, k is a proportionality factor that is a value between 0 and 1 (and may include the values 0 or 1), Qₚ is the second signal, and Q_{pcal} is the second signal at the calibration time point.

Exemplary methods to enhances transport of the analyte across a skin or mucosal surface of the subject include, but are not limited to, iontophoresis, sonophoresis, suction, electroporation, thermal poration, laser poration, use of microporation, use of microneedles, use of microfine lances, skin permeabilization, chemical permeation enhancers, use of laser devices, and combinations thereof.

The one or more microprocessors of the present invention in some embodiments further comprise programming to control operating a first sensing device that provides the first signal and operating a second sensing device that provides the second signal. Further, in some embodiments the one or more microprocessors of the present invention comprise programming to control operating a first sampling device (e.g., where the sampling device employs iontophoresis) that provides the first sample.

These and other embodiments of the present invention will readily occur to those of ordinary skill in the art in view of the disclosure herein.

### BRIEF DESCRIPTION OF THE FIGURES

FIG. 1 presents a schematic of an exploded view of exemplary components comprising one embodiment of a standard AutoSensor assembly for use in Cygnus' GlucoWatch biographer systems, having two active collection reservoirs (i.e., collection reservoirs through which iontophoretic current is passed) for use in an analyte monitoring device. The AutoSensor components include two biosensor/iontophoretic electrode assemblies, 104 and 106, each of which have an annular iontophoretic electrode, respectively indicated at 108 and 110, which encircles a biosensor electrode 112 and 114. The electrode assemblies 104 and 106 are printed onto a polymeric substrate 116 which is maintained within a sensor tray 118. A collection reservoir assembly 120 is arranged over the electrode assemblies, wherein the collection reservoir assembly comprises two hydrogel inserts 122 and 124 retained by a gel retaining layer 126 and mask layer 128. Additionally, release liners may be included in the assembly, for example, a patient liner 130, and a plow-fold liner 132. In one embodiment, the electrode assemblies comprise bimodal electrodes.

FIGS. 2-11 present a series of schematic diagrams of two exemplary AutoSensor assemblies each having a third, passive collection reservoir, wherein different layers are illustrated in each figure.

FIG. 2 presents a schematic diagram of screen printed sensor inks on a sensor substrate. In the figure, platinum (Pt) ink is shown in light grey, silver (Ag) ink is shown in black, and silver chloride (AgCl) ink is shown in dark grey. The outline geometry of the sensor substrate is shown.

FIG. 3 presents a schematic diagram of a dielectric layer added on top of the printed sensor.

FIG. 4 presents a skin-side schematic diagram that shows the sensors after wrapping around the tray and staking or otherwise adhering the sensor to the tray.

FIG. 5 presents a schematic diagram of the side facing away from the skin corresponding to FIG. 4.

FIG. 6 presents a schematic diagram of the gel retaining layer (GRL) or corral attached to the sensor.

FIG. 7 presents a schematic diagram of the hydrogel discs (collection reservoirs) placed in position.

FIG. 8 presents a schematic diagram of a mask layer placed in position over the sensor.

FIG. 9 presents a schematic diagram of a removable plowfold layer that separates the hydrogel from the silver/silver-chloride electrodes during storage.

FIG. 10 presents a schematic diagram of a removable patient liner that covers the adhesive on the mask and the hydrogel.

FIG. 11 presents a schematic diagram of all the layers simultaneously that comprise a total AutoSensor assembly.

FIG. 12 presents a plot including data from all six subjects for active versus passive adjusted nanoCoulomb (nC) signals for sweat and non-sweat events. In the figure, Δ nC from condition 1 (with iontophoresis, Δ nC Active=−Qat+Qas) is presented on the y-axis, Δ nC from condition 2 (no iontophoresis, Δ nC Passive=Qpt+Qps) is presented on the x-axis, X represents sensor A sweat values, +represents sensor B sweat values, O represents sensor A non-sweat values, and Δ represents sensor B non-sweat values. The plot is active versus passive change in nC signal for sweat and non-sweat events. The equation representing the linear regression is as follows: y=0.9995x+179.16, with an R²=0.5822.

FIG. 13 presents a plot including data from all six subjects for active versus passive adjusted from calibration (CAL) nC signals for sweat and non-sweat events. In the figure, Δ nC from condition 1 (with iontophoresis, Δ nC Active=Qat+Qas) is presented on the y-axis, Δ nC from condition 2 (no iontophoresis, Δ nC Passive Adjusted from CAL=Qp−Qpcal) is presented on the x-axis, X represents sensor A sweat values, +represents sensor B sweat values, o represents sensor A non-sweat values, and Δ represents sensor B non-sweat values. The plot is active versus passive change in nC signal for sweat and non-sweat events. The equation representing the linear regression is as follows: y=0.8951x +229.99, with an R²=0.524.

FIG. 14 presents a bar graph showing Mean Absolute Relative Error (MARE) of Biographer glucose readings compared to blood glucose measurements at different skin conductivity values.

FIG. 15 presents an illustrative plot of nC signal at the cathode (Qa) for an active collection reservoir/sensing electrode (i.e., extraction with iontophoresis was performed) on the y-axis, and elapsed time on the x-axis. The dots represent individual nC signals and the line represents a best-fit linear regression of the nC data points. The “x”s represent nC signals at time points associated with perspiration events.

FIG. 16 presents an illustrative plot of nC signal at the cathode (Qp) for a passive collection reservoir/sensing electrode (i.e., no extraction with iontophoresis was performed) on the y-axis, and elapsed time on the x-axis. The dots represent individual nC signals and the line represents a best-fit linear regression of the nC data points. The “x”s represent nC signals at time points associated with perspiration events.

FIG. 17 presents an illustrative plot of nC signal at the cathode (Qp) for a passive collection reservoir/sensing electrode (i.e., no extraction with iontophoresis was performed) on the y-axis, and elapsed time on the x-axis. The line represents the nC signal at calibration (Qpcal). The “x”s represent nC signals at time points associated with perspiration events.

FIG. 18 illustrates an example of a Qpthresh (threshold value for the passive signal, above which, prediction of the blood glucose value is skipped), which is shown by the vertical dotted line. The data in this figure correspond to the data shown in FIG. 13.

FIG. 19 illustrates an example of a reference collection reservoir (“reference gel” in the figure).

### DETAILED DESCRIPTION OF THE INVENTION

The practice of the present invention will employ, unless otherwise indicated, conventional methods of diagnostics, chemistry, biochemistry, electrochemistry, statistics, and pharmacology, within the skill of the art in view of the teachings of the present specification. Such conventional methods are explained fully in the literature.

All patents, publications, and patent applications cited in this specification are herein incorporated by reference as if each individual patent, publication, or patent application was specifically and individually indicated to be incorporated by reference in its entirety for all purposes.

1.0.0 Definitions

It is to be understood that the terminology used herein is for the purpose of describing particular embodiments only, and is not intended to be limiting. As used in this specification and the appended claims, the singular forms “a,” “an” and “the” include plural referents unless the context clearly dictates otherwise. Thus, for example, reference to “a reservoir” includes a combination of two or more such reservoirs, reference to “an analyte” includes one or more analytes, mixtures of analytes, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

The term “microprocessor” refers to a computer processor contained on an integrated circuit chip, such a processor may also include memory and associated circuits. A microprocessor may also include programmed instructions to execute or control selected functions; computational methods, switching, etc. Microprocessors and associated devices are commercially available from a number of sources, including, but not limited to, Cypress Semiconductor Corporation, San Jose, Calif.; IBM Corporation, White Plains, N.Y.; Applied Microsystems Corporation, Redmond, Wash.; Intel Corporation, Santa Clara, Calif.; and National Semiconductor, Santa Clara, Calif.

The terms “analyte” and “target analyte” are used to denote any physiological analyte of interest that is a specific substance or component that is being detected and/or measured in a chemical, physical, enzymatic, or optical analysis. A detectable signal (e.g., a chemical signal or electrochemical signal) can be obtained, either directly or indirectly, from such an analyte or derivatives thereof Furthermore, the terms “analyte” and “substance” are used interchangeably herein, and are intended to have the same meaning, and thus encompass any substance of interest. In preferred embodiments, the analyte is a physiological analyte of interest, for example, glucose, or a chemical that has a physiological action, for example, a drug or pharmacological agent.

A “sampling device,” “sampling mechanism,” or “sampling system” refers to any device and/or associated method for obtaining a sample from a biological system for the purpose of determining the amount or concentration of an analyte of interest in the biological system. Such “biological systems” include any biological system from which the analyte of interest can be extracted, including, but not limited to, blood, interstitial fluid, perspiration and tears. Further, a “biological system” includes both living and artificially maintained systems. The term “sampling” method refers to extraction of a substance from the biological system, generally across a membrane such as the stratum corneum or mucosal membranes, wherein said sampling is invasive, minimally invasive, semi-invasive or non-invasive. The membrane can be natural or artificial, and can be of plant or animal nature, such as natural or artificial skin, blood vessel tissue, intestinal tissue, and the like. A sampling mechanism may be in operative contact with a “reservoir,” or “collection reservoir,” wherein the sampling mechanism is used for extracting the analyte from the biological system into the reservoir to obtain the analyte in the reservoir. Alternately, a sampling device or sampling method may be used to treat the skin or mucosal surface, the sampling device removed, and sample collected into a collection reservoir that is typically in operative contact with a sensing device. Non-limiting examples of sampling methods include iontophoresis (including reverse iontophoresis and electroosmosis), sonophoresis, microdialysis, suction, electroporation, thermal poration, use of microporation (e.g., by laser or thermal ablation), biolistic (e.g., using particles accelerated to high speeds), use of microneedles, use of microfine lances, microfine cannulas, skin permeabilization, chemical permeation enhancers, use of laser devices, and combinations thereof. These sampling methods are well known in the art, for example, iontophoresis (see, e.g., PCT International Publication Nos. WO 97/24059, WO 96/00110, and WO 97/10499; European Patent Application No. EP 0942 278; U.S. Pat. Nos. 5,771,890, 5,989,409, 5,735,273, 5,827,183, 5,954,685, 6,023,629, 6,298,254, 6,687,522, 5,362,307, 5,279,543, 5,730,714, 6,542,765, and 6,714,815), sonophoresis (see, e.g., Chuang H, et al., Diabetes Technology and Therapeutics, 6(1):21-30, 2004; U.S. Pat. Nos. 6,620,123, 6,491,657, 6,234,990, 5,636,632, and 6,190,315; PCT International Publication No. WO 91/12772; and Merino, G, et al, J Pharm Sci. 2003 June;92(6):1125-37), suction (see, e.g., U.S. Pat. No. 5,161,532), electroporation (see, e.g., U.S. Pat. No. 6,512,950, and 6,022,316), thermal poration (see, e.g., U.S. Pat. No. 5,885,211), use of microporation (see, e.g., U.S. Pat. No. 6,730,028, 6,508,758, and 6,142,939), use of microneedles (see, e.g., U.S. Pat. No. 6,743,211), use of microfine lances (see, e.g., U.S. Pat. No. 6,712,776), skin permeabilization (see, e.g., Ying Sun, Transdermal and Topical Drug Delivery Systems, Interpharm Press, Inc., 1997, pages 327-355), chemical permeation enhancers (see, e.g., U.S. Pat. No. 6,673,363), and use of laser devices (see, e.g., Gebhard S, et al., Diabetes Technology and Therapeutics, 5(2), 159-166, 2003; Jacques et al. (1978) J. Invest. Dermatology 88:88-93; PCT International Publication Nos. WO 99/44507, WO 99/44638, and WO 99/40848).

The term “physiological fluid” refers to any desired fluid to be sampled, and includes, but is not limited to, blood, cerebrospinal fluid, interstitial fluid, semen, sweat, saliva, urine and the like.

The term “artificial membrane” or “artificial surface” refers to, for example, a polymeric membrane, or an aggregation of cells of monolayer thickness or greater which are grown or cultured in vivo or in vitro, wherein said membrane or surface functions as a tissue of an organism but is not actually derived, or excised, from a pre-existing source or host.

A “monitoring system,” “analyte monitoring system,” or “analyte monitoring device” refers to a system useful for obtaining frequent measurements of a physiological analyte present in a biological system (e.g., analyte amount or concentration in blood or interstitial fluid). Such a system typically comprises, but is not limited to, a sensing device and one or more microprocessors in operative combination with the sensing device, or a sampling device, a sensing device, and one or more microprocessors in operative combination with the sampling device and/or the sensing device.

A “measurement cycle” typically comprises sensing of an analyte in a sample, for example, using a sensing device, to provide a measured signal, for example, a measured signal response curve. Typically a series of measurement cycles provides a series of measured signals. In some embodiments, the measurement cycle further comprises extraction of an analyte from a subject, using, for example, a sampling device. Accordingly, in some embodiments, a measurement cycle comprises one or more sets of extraction and sensing.

The term “frequent measurement” refers to a series of two or more measurements obtained from a particular biological system, which measurements are obtained using a single device maintained in operative contact with the biological system over a time period in which a series of measurements (e.g., second, minute or hour intervals) is obtained. The term thus includes continual and continuous measurements.

The term “subject” encompasses any warm-blooded animal, particularly including a member of the class Mammalia such as, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

The term “transdermal” includes both transdermal and transmucosal techniques, i.e., extraction of a target analyte across skin, for example, stratum corneum, or mucosal tissue. Aspects of the invention, which are described herein in the context of “transdermal,” unless otherwise specified, are meant to apply to both transdermal and transmucosal techniques.

The term “transdermal extraction” or “transdermally extracted” refers to any sampling method, which entails extracting and/or transporting an analyte across skin or mucosal tissue. The term thus includes extraction of an analyte using methods including, but not limited to, iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, use of biolistics and combinations thereof. Transdermal extraction methods typically enhance transport of the analyte across a skin (e.g., stratum corneum) or mucosal surface, wherein the enhancement is relative to analyte transport in the absence of an applied transdermal extraction method.

The term “iontophoresis” refers to a method for transporting substances across tissue by way of an application of electrical energy to the tissue. In conventional iontophoresis, a reservoir is provided at the tissue surface to serve as a container of (or to provide containment for) material to be transported. Iontophoresis can be carried out using standard methods known to those of skill in the art, for example by establishing an electrical potential using a direct current (DC) between fixed anode and cathode “iontophoretic electrodes,” alternating a direct current between anode and cathode iontophoretic electrodes, or using a more complex waveform such as applying a current with alternating polarity (AP) between iontophoretic electrodes (so that each electrode is alternately an anode or a cathode). For example, see U.S. Pat. Nos. 5,771,890, 6,023,629, 6,298,254, 6,687,522, and PCT International Publication No. WO 96/00109.

The term “reverse iontophoresis” refers to the movement of a substance from a biological fluid across a membrane by way of an applied electric potential or current. In reverse iontophoresis, a reservoir is provided at the tissue surface to receive the extracted material, as used in GlucoWatch biographer monitoring devices.

“Electroosmosis” refers to the movement of a substance through a membrane by way of an electric field-induced convective flow. The terms iontophoresis, reverse iontophoresis, and electroosmosis, will be used interchangeably herein to refer to movement of any ionically charged or uncharged substance across a membrane (e.g., an epithelial membrane) upon application of an electric potential to the membrane through an ionically conductive medium.

The term “sensing device” or “sensing mechanism” encompasses any device that can be used to measure the concentration or amount of an analyte, or derivative thereof, of interest. Preferred sensing devices for detecting analytes (e.g., in blood or interstitial fluid) generally include electrochemical devices, optical and chemical devices and combinations thereof. Examples of electrochemical devices include the Clark electrode system (see, e.g., Updike, et al., (1967) Nature 214:986-988), and other amperometric, coulometric, or potentiometric electrochemical devices, as well as, optical methods, for example UV detection or infrared detection (e.g., U.S. Pat. No. 5,747,806). For example, U.S. Pat. No. 5,267,152 describes a noninvasive technique of measuring blood glucose concentration using near-IR radiation diffuse-reflection laser spectroscopy. Near-IR spectrometric devices are also described in U.S. Pat. Nos. 5,086,229, 5,747,806, and 4,975,581. Additional examples include electrochemical analyte sensors, for example, as described in U.S. Pat. Nos. 6,134,461, 6,175,752, 6,587,705, and 6,736,777. A sensing device typically provides a detectable “signal” related to analyte amount or concentration in, for example, a subject or sample obtained from a subject. Typical signals include, but are not limited to, electrical signals (e.g., amperometric or coulometric signals), optical signals (e.g., detection of specific emitted wavelengths or absorption patterns, or fluorescence), and chemical signals (e.g., calorimetric signals). Such signals may be used directly or further processed to obtain a related analyte measurement value, for example, using the methods described herein.

A “biosensor” or “biosensor device” includes, but is not limited to, a “sensor element” that includes, but is not limited to, a “biosensor electrode” or “sensing electrode” or “working electrode” which refers to the electrode that is monitored to determine the amount of electrical signal at a point in time or over a given time period, which signal is then correlated with the concentration of a chemical compound. The sensing electrode comprises a reactive surface that converts the analyte, or a derivative thereof, to electrical signal. The reactive surface can be comprised of any electrically conductive material such as, but not limited to, platinum-group metals (including, platinum, palladium, rhodium, ruthenium, osmium, and iridium), nickel, copper, and silver, as well as, oxides, and dioxides, thereof, and combinations or alloys of the foregoing, which may include carbon as well. Some biosensor electrode embodiments are described in EP 0 942 278, GB 2 335 278, U.S. Pat. Nos. 6,042,751, 6,587,705, 6,736,777, published U.S. Patent Application No. 20030155557, and PCT International Publication No. WO 03/054070. Some catalytic materials, membranes, and fabrication technologies suitable for the construction of amperometric biosensors are also described by Newman, J. D., et al.(1995) Analytical Chemistry 67:4594-4599. In some embodiments, the biosensor comprises a sensing element (e.g., a platinum-based sensing electrode) and one or more enzymes to facilitate detection of analyte. For example, when the analyte is glucose, glucose oxidase may be used. Additional enzymes my be used as well, for example, glucose oxidase and a mutarotase enzyme.

The “sensor element” can include components in addition to the sensing electrode, for example, it can include a “reference electrode” and a “counter electrode.” The term “reference electrode” is used to mean an electrode that provides a reference potential, for example, a potential can be established between a reference electrode and a working electrode. The term “counter electrode” is used to mean an electrode in an electrochemical circuit that acts as a current source or sink to complete the electrochemical circuit. Although it is not essential that a counter electrode be employed where a reference electrode is included in the circuit and the electrode is capable of performing the function of a counter electrode, it is preferred to have separate counter and reference electrodes because the reference potential provided by the reference electrode is most stable when it is at equilibrium. If the reference electrode is required to act further as a counter electrode, the current flowing through the reference electrode may disturb this equilibrium. Consequently, separate electrodes functioning as counter and reference electrodes are preferred.

In one embodiment, the “counter electrode” of the “sensor element” comprises a “bimodal electrode.” The term “bimodal electrode” typically refers to an electrode which is capable of functioning non-simultaneously as, for example, both the counter electrode (of the “sensor element”) and the iontophoretic electrode (of the “sampling mechanism”) as described, for example, U.S. Pat. No. 5,954,685.

The terms “reactive surface” and “reactive face” are used interchangeably herein to mean the catalytic surface of the sensing electrode. In some embodiments, the reactive surface is (1) in contact with the surface of an ionically conductive material which contains an analyte or through which an analyte, or a derivative thereof, flows from a source thereof; (2) comprised of a catalytic material (e.g., a platinum group metal, platinum, palladium, rhodium, ruthenium, or nickel and/or oxides, dioxides and combinations or alloys thereof) or a material that provides sites for electrochemical reaction; (3) converts a chemical signal (e.g., hydrogen peroxide) into an electrical signal (e.g., an electrical current); and (4) defines the electrode surface area that, when composed of a reactive material, is sufficient to drive the electrochemical reaction at a rate sufficient to generate a detectable, reproducibly measurable, electrical signal when an appropriate electrical bias is supplied, that is correlatable with the amount of analyte present in the electrolyte. Further, a polymeric membrane may be used at, for example, the electrode surface to block or inhibit access of interfering species to the reactive surface of the electrode.

An “ionically conductive material” refers to any material that provides ionic conductivity, and through which electrochemically active species can diffuse. The ionically conductive material can be, for example, a solid, liquid, or semi-solid (e.g., in the form of a gel) material that contains an electrolyte, which can be composed primarily of water and ions (e.g., sodium chloride), and generally comprises 50% or more water by weight. The material can be in the form of a hydrogel, a sponge or pad (e.g., soaked with an electrolytic solution), or any other material that can contain an electrolyte and allow passage of electrochemically active species, especially the analyte of interest. Some exemplary hydrogel formulations are described in PCT International Publication Nos. WO 97/02811 and WO 00/64533, as well as EP 0 840 597 B1, U.S. Pat. No. 6,615,078, and published U.S. Patent Application No. 20040062759. In some embodiments, the ionically conductive material comprises a biocide. For example, during manufacture of an AutoSensor assembly, one or more biocides may be incorporated into the ionically conductive material. Biocides of interest include, but are not limited to, compounds such as chlorinated hydrocarbons; organometallics; metallic salts; organic sulfur compounds; phenolic compounds (including, but not limited to, a variety of Nipa Hardwicke Inc. liquid preservatives registered under the trade names Nipastat®, Nipaguard®, Phenosept®, Phenonip®, Phenoxetol®, and Nipacide®); quaternary ammonium compounds; surfactants and other membrane-disrupting agents (including, but not limited to, undecylenic acid and its salts), combinations thereof, and the like.

“Hydrophilic compound” refers to a monomer that attracts, dissolves in, or absorbs water. The hydrophilic compounds for use according to the invention are one or more of the following: carboxy vinyl monomer, a vinyl ester monomer, an ester of a carboxy vinyl monomer, a vinyl amide monomer, a hydroxy vinyl monomer, a cationic vinyl monomer containing an amine or a quaternary ammonium group. The monomers can be used to make the polymers or copolymers including, but not limited to, polyethylene oxide (PEO), polyvinyl alcohol, polyacrylic acid, and polyvinyl pyrrolidone (PVP).

The term “buffer” refers to one or more components which are added to a composition in order to adjust or maintain the pH of the composition.

The term “electrolyte” refers to a component of the ionically conductive medium which allows an ionic current to flow within the medium. This component of the ionically conductive medium can be one or more salts or buffer components, but is not limited to these materials.

The term “collection reservoir” is used to describe any suitable containment method or device for containing a sample extracted from a biological system. For example, the collection reservoir can be a receptacle containing a material that is ionically conductive (e.g., water with ions therein), or alternatively it can be a material, such as a sponge-like material or hydrophilic polymer, used to keep the water in place. Such collection reservoirs can be in the form of a sponge, porous material, or hydrogel (e.g., in the shape of a disk or pad). Hydrogels are typically referred to as “collection inserts.” Other suitable collection reservoirs include, but are not limited to, tubes, vials, strips, capillary collection devices, cannulas, and miniaturized etched, ablated or molded flow paths.

A “collection insert layer” is a layer of an assembly or laminate comprising one or more collection reservoir (or collection insert) located, for example, between a mask layer and a retaining layer.

A “laminate” refers to structures comprised of, at least, two bonded layers. The layers may be bonded by welding or through the use of adhesives. Examples of welding include, but are not limited to, the following: ultrasonic welding, heat bonding, and inductively coupled localized heating followed by localized flow.

Examples of common adhesives include, but are not limited to, chemical compounds such as, cyanoacrylate adhesives, and epoxies, as well as adhesives having such physical attributes as, but not limited to, the following: pressure sensitive adhesives, thermoset adhesives, contact adhesives, and heat sensitive adhesives.

A “collection assembly” refers to structures comprised of several layers, where the assembly includes at least one collection insert layer, for example a hydrogel. An example of a collection assembly as referred to in the present invention is a mask layer, collection insert layer, and a retaining layer where the layers are held in appropriate functional relationship to each other but are not necessarily a laminate (i.e., the layers may not be bonded together. The layers may, for example, be held together by interlocking geometry or friction).

The term “mask layer” refers to a component of a collection assembly that is substantially planar and typically contacts both the biological system and the collection insert layer. See, for example, U.S. Pat. Nos. 5,827,183, 5,735,273, 6,141,573, 6,201,979, 6,370,410, and 6,529,755.

The term “gel retaining layer” or “gel retainer” refers to a component of a collection assembly that is substantially planar and typically contacts both the collection insert layer and the electrode assembly. See, for example, U.S. Pat. Nos. 6,393,318, 6,341,232, and 6,438,414.

The term “support tray” typically refers to a rigid, substantially planar platform and is used to support and/or align the electrode assembly and the collection assembly. The support tray provides one way of placing the electrode assembly and the collection assembly into the sampling system.

An “AutoSensor assembly” refers to a structure generally comprising a mask layer, collection insert layer, a gel retaining layer, an electrode assembly, and a support tray. The AutoSensor assembly may also include liners where the layers are held in approximate, functional relationship to each other. Exemplary collection assemblies and AutoSensor structures are described, for example, U.S. Pat. Nos. 5,827,183, 5,735,273, 6,141,573, 6,201,979, 6,370,410, 6,393,318, 6,341,232, 6,438,414, and 6,529,755. One such AutoSensor assembly is available from Cygnus, Inc., Redwood City, Calif. These exemplary collection assemblies and AutoSensors may be modified by use of the ionically conductive materials (e.g., hydrogels) of the present invention. The mask and retaining layers are preferably composed of materials that are substantially impermeable to the analyte (chemical signal) to be detected; however, the material can be permeable to other substances. By “substantially impermeable” is meant that the material reduces or eliminates chemical signal transport (e.g., by diffusion). The material can allow for a low level of chemical signal transport, with the proviso that chemical signal passing through the material does not cause significant edge effects at the sensing electrode.

The terms “about” or “approximately” when associated with a numeric value refers to that numeric value plus or minus 10 units of measure (i.e. percent, grams, degrees or volts), preferably plus or minus 5 units of measure, more preferably plus or minus 2 units of measure, most preferably plus or minus 1 unit of measure.

By the term “printed” is meant a substantially uniform deposition of a conductive polymer composite film (e.g., an electrode ink formulation) onto one surface of a substrate (i.e., the base support). It will be appreciated by those skilled in the art that a variety of techniques may be used to effect substantially uniform deposition of a material onto a substrate, for example, Gravure-type printing, extrusion coating, screen coating, spraying, painting, electroplating, laminating, or the like.

The term “physiological effect” encompasses effects produced in the subject that achieve the intended purpose of a therapy. In preferred embodiments, a physiological effect means that the symptoms of the subject being treated are prevented or alleviated. For example, a physiological effect would be one that results in the prolongation of survival in a patient.

“Parameter” refers to an arbitrary constant or variable so appearing in a mathematical expression that changing it give various cases of the phenomenon represented (McGraw-Hill Dictionary of Scientific and Technical Terms, S. P. Parker, ed., Fifth Edition, McGraw-Hill Inc., 1994). In the context of GlucoWatch biographer monitoring devices, a parameter is a variable that influences the value of the blood glucose level as calculated by an algorithm.

“Decay” refers to a gradual reduction in the magnitude of a quantity, for example, a current detected using a sensor electrode where the current is correlated to the concentration of a particular analyte and where the detected current gradually reduces but the concentration of the analyte does not.

“Screens” or “Screening” refer to applying one or more predetermined criteria to data, for example, a signal, to determine whether the data conform to the criteria. “Skip” or “skipped” signals refer to data that do not conform to predetermined criteria (e.g., error-associated criteria as described in U.S. Pat. No. 6,233,471 and 6,595,919). A skipped reading, signal, or measurement value typically has been rejected (i.e., a “skip error” generated) as not being reliable or valid because it does not conform with data integrity checks, for example, where a signal is subjected to one or more data screens that invalidate incorrect signals based on one or more detected parameters indicative of a poor or incorrect signal. Further exemplary screens are described herein, for example, a threshold may be set (e.g., Qpthresh) wherein an active signal obtained for substantially the same time period as a passive signal, wherein the passive signal is above a certain value, is skipped or corrected.

A “future time point” refers to the time point in the future at which the concentration of the analyte of interest or another parameter value is predicted. In preferred embodiments, this term refers to a time point that is one time interval ahead, where a time interval is the amount of time between sampling and sensing events.

“Active” collection reservoirs/sensing devices (e.g., active collection reservoir/sensing electrode) refer to the application of any transdermal sampling method to a subject to provide a sample comprising analyte, wherein the method enhances transdermal transport (skin flux) of analyte into the collection reservoir/sensing device in order to obtain an analyte measurement value. Exemplary transdermal sampling methods of enhancing transdermal transport are described herein including, but not limited to, iontophoresis, sonophoresis, suction, electroporation, thermal poration, use of microporation (e.g., by laser or thermal ablation), use of microneedles, use of microfine lances, skin permeabilization, chemical permeation enhancers, and use of laser devices. In contrast, “passive” collection reservoirs/sensing devices (e.g., passive collection reservoir/sensing electrode) refer to obtaining a sample that may comprise analyte, however no method is employed to enhance transdermal transport of analyte into the collection reservoir/sensing device in order to obtain an analyte measurement value. A passive collection reservoir/sensing device may, for example, provide a sample obtained as a result of transdermal passive diffusion into the collection reservoir/sensing device and any accompanying collection of sweat. A passive collection reservoir/sensing device may also, for example, provide information about signal obtained using the sensing device that is related to temperature fluctuations.

1.1.0 GlucoWatch Biographer Monitoring Devices

The terms “GlucoWatch biographer” and “GlucoWatch G2 biographer” refer to two exemplary devices in a line of GlucoWatch® (Cygnus, Inc., Redwood City, Calif.) biographer monitoring devices developed and manufactured by Cygnus, Inc., Redwood City, Calif.

GlucoWatch biographers analyte monitoring devices provide automatic, frequent, and noninvasive glucose measurements. The first-generation device, the GlucoWatch biographer, provided up to 3 readings per hour for as long as 12 hours after a 3-hour warm-up period and a single blood glucose (BG) measurement for calibration. The second-generation device, the GlucoWatch G2 biographer, provides up to six readings per hour for as long as 13 hours after a single BG measurement for calibration. These devices utilize reverse iontophoresis to extract glucose through the skin. The glucose is then detected by an amperometric biosensor in the AutoSensor. GlucoWatch biographer monitoring devices are small devices, typically worn on the forearm, that contain sampling and detection circuitry, and a digital display. Clinical trials on subjects with Type 1 and Type 2 diabetes have shown excellent correlation between GlucoWatch biographer readings and serial finger-stick BG measurements (see, e.g., Garg, S. K., et al., Diabetes Care 22, 1708 (1999); Tamada, J. A., et al., JAMA 282, 1839 (1999)). However, the first-generation GlucoWatch biographer measurement period was limited to up to 12 hours, due to decay of the biosensor signal during use. The second-generation device extends the measurement period to up to 13 hours.

GlucoWatch biographer monitoring devices have several advantages. Clearly their non-invasive and non-obtrusive nature encourages more glucose testing among people with diabetes. Of greater clinical relevance is the frequent nature of the information provided. GlucoWatch biographer monitoring devices provide the more frequent monitoring desired by physicians in an automatic, non-invasive, and user-friendly manner. The automatic nature of the systems also allow monitoring to continue even while the patient is sleeping or otherwise unable to test. The GlucoWatch biographer and GlucoWatch G2 biographer are the only frequent, automatic, and non-invasive glucose-monitoring devices approved by the U.S. Food and Drug Administration and commercially available.

1.1.1 Device Description of GlucoWatch Biographer Monitoring Devices

GlucoWatch biographer monitoring devices contain the electronic components that supply iontophoretic current and controls current output and operating time. They also control the biosensor electronics, as well as receive, process, display and store data. Data can also be uploaded from GlucoWatch biographer monitoring devices to a personal computer, a computer network, personal digital assistant device, etc. They have bands to help secure them to sites on the forearm.

The AutoSensor is a consumable part of the devices that provides up to 13 hours of continuous glucose measurement (in the second-generation device). The AutoSensor is discarded after each wear period. It fits into the back of a GlucoWatch biographer monitoring device and contains electrodes for delivery of iontophoretic current, sensor electrodes for sensing the glucose signal, and glucose-oxidase-containing hydrogel pads for glucose collection and conversion to hydrogen peroxide. There are two gel/electrode sets on each AutoSensor, denoted as A and B.

Iontophoresis utilizes the passage of a constant low-level electrical current between two electrodes applied onto the surface of the skin. This technique has been used, for example, to deliver transdermally ionic (charged) drugs (Sinh J., et al., Electrical properties of skin, in “Electronically controlled drug delivery,” Berner B, and Dinh S M, eds., Boca Raton, La.: CRC Press (1998), pp. 47-62.). On the other hand, electrolyte ions in the body can also act as the charge carriers and can lead to extraction of substances from the body outward through the skin. This process is known as “reverse iontophoresis” or iontophoretic extraction (Rao, G. et al., Pharm. Res. 10, 1751 (2000)). Because skin has a net negative charge at physiological pH, positively charged sodium ions are the major current carriers across the skin. The migration of sodium ions toward the iontophoretic cathode creates an electro-osmotic flow, which carries neutral molecules by convection. However, only compounds with small molecular weight pass through the skin, so that, for example, no proteins are extracted. Moreover, major interfering species (e.g., ascorbate and urate) are collected at anode. As a result of these unique charge and size exclusion properties of reverse iontophoresis, glucose is preferentially extracted at the cathode, and the obtained sample is very clean. This is in contrast to implantable glucose monitoring devices (Gross, T. M., Diabetes Technology and Therapeutics 2, 49 (2000); Meyerhoff, C., et al., Diabetologia, 35, 1087 (1992); Bolinder, J., et al., Diabetes Care 20, 64 (1997)) for which ascorbate and urate (as well as some proteins) are known to produce an interfering signal.

The feasibility of iontophoretic glucose extraction for glucose monitoring was demonstrated in human subjects (Tamada, J. A., et al., Nat. Med. 1, 1198 (1995)). In feasibility studies with human subjects, glucose transport correlated well with blood glucose (BG) in a linear manner. However, the sensitivity (i.e., the amount of glucose extracted) varied among individuals and skin sites (Tamada, J. A., et al., Nat. Med. 1, 1198 (1995)). A single-point calibration was found to compensate for this variability. Reverse iontophoresis yielded micromolar concentrations of glucose in the receiver solution, which was about three orders of magnitude less than that found in blood.

To accurately measure this small amount of glucose, GlucoWatch biographer monitoring devices utilize an amperometric biosensor (Tierney, M. J., et al., Clin. Chem. 45, 1681 (1999)). The glucose oxidase (GOx) enzyme in hydrogel disks (where glucose is collected via reverse iontophoresis) catalyzes the reaction of glucose with oxygen to produce gluconic acid and hydrogen peroxide,

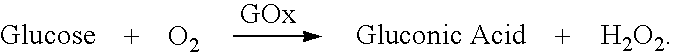

Glucose exists in two forms: α and β-glucose, which differ only in the position of a hydroxyl group. At equilibrium (also in blood and in interstitial fluid), the two forms are in proportion of about 37% α and about 63% β. As glucose enters the hydrogel, it diffuses throughout, and only the β-form of glucose reacts with the glucose oxidase enzyme. As β-form is depleted, the α-form then converts (mutarotates) to the β-form. The products of the glucose oxidase reaction (hydrogen peroxide and gluconic acid) also diffuse throughout the gel. Finally, hydrogen peroxide (H₂O₂) is detected at a platinum-containing working electrode in the sensor via the electro-catalytic oxidation reaction,
H₂O₂→O₂+2 H⁺+2 e⁻
producing measurable electrical current, and regenerating O₂. Thus, ideally, for every glucose molecule extracted, two electrons are transferred to the measurement circuit. Integration over time of the resulting electric current leads to the total charge liberated at the electrode, and the latter is correlated to the amount of glucose collected through the skin.

The structure of the commercial second-generation device is very similar to the first-generation device. Extraction and detection are achieved using two hydrogel pads (A and B) placed against the skin. The side of each pad away from the skin is in contact with an electrode assembly containing two sets of iontophoretic and sensing elements. The two electrode sets complete the iontophoretic circuit. During operation, one iontophoretic electrode is cathodic and the other anodic, enabling the passage of current through the skin. As a consequence, glucose and other substances are collected in the hydrogel pads during the iontophoretic extraction period. The iontophoretic time interval is adjusted to minimize skin irritation and power requirements, yet extract sufficient glucose for subsequent detection. It has been found that a useful time for extraction of glucose is about three minutes.

On the side of each hydrogel pad, away from the skin and adjacent to the annular iontophoretic electrode, are the sensing electrodes. There are two sensing electrodes, noted as sensor A and B. These circular sensing electrodes are composed of a platinum composite, and are activated by applying a potential of 0.3-0.8 V (relative to a Ag/AgCl reference electrode). At these applied potentials, a current is then generated from the reaction of H₂O₂ (generated from extracted glucose) that has diffused to the platinum sensor electrode.

1.1.2 Device Operation of GlucoWatch Biographer Monitoring Devices

Each 20 minute glucose measurement cycle consists of three minutes of extraction, and seven minutes of biosensor activation, followed by three minutes of extraction at the opposite iontophoresis current polarity, and seven additional minutes of biosensor activation.

In the first half-cycle, glucose is collected in the hydrogel at the iontophoretic cathode (Sensor B). As the glucose is collected, it reacts with the glucose oxidase in the hydrogel to produce hydrogen peroxide. At the end of the three-minute collection period, the iontophoretic current is stopped, and the biosensors activated for seven minutes to measure the accumulated H₂O₂. This period is chosen so that the vast majority of the extracted glucose is converted to H₂O₂, and that the vast majority of this peroxide diffuses to the platinum electrode, and subsequently oxidizes to generate a current. Because the underlying physical and chemical processes (including, but not limited to, diffusion, glucose mutarotation, and electro-catalytic oxidation reaction at the sensing electrodes) are rather slow, not all of the extracted glucose and H₂O₂ is consumed during the seven-minute measurement cycle. However, the integrated current (or charge) signal over this seven-minute interval is sufficiently large and remains proportional to the total amount of glucose that entered the hydrogel pad during the iontophoresis interval. In the process of detection, majority of H₂O₂ is depleted. This cleans out the hydrogel to be ready for the next collection period. Moreover, before sensor B will be collecting and measuring glucose again, it has to act as an iontophoretic anode first. The extraction-sensing cycles have been designed so that there will be no peroxide left in the hydrogel after this period. During the initial three-minute period, there is also-extraction at the anode (sensor A), primarily of anionic species such as urate and ascorbate. These electrochemically active species are also purged from the anodic-reservoir during the seven-minute biosensor period.

In the second half-cycle of the measurement cycle, the iontophoretic polarity is reversed, so that glucose collection at the cathode occurs in the second reservoir (sensor A), and the anionic species are collected in the first reservoir (sensor B). The biosensor is again activated to measure glucose at the cathode (now sensor A) and to purge electrochemically active species for the anode (sensor B). The combined twenty-minute process is repeated to obtain each subsequent glucose reading.

The raw data for each half-cycle are collected for both A and B sensors as 13 discrete current values measured as functions of time over the seven minutes (providing a measured signal response curve). When the sensor circuits are activated in the cathodic cycle, H₂O₂ (converted from glucose) reacts with the platinum electrode to produce a current, which monotonically declines with time over the seven-minute detection cycle. A current signal of similar shape is also generated in the anodic cycle (curve with data points represented with diamonds). This signal is due, in large part, to ascorbic and uric acids. In both cases the current transients come down to a background of approximately 180 nA rather than zero. The background current, termed the baseline background, does not vary much over time, indicating that it is likely the result of the sum of a number of low concentration species. In order to extract the glucose-related signal only, the background is subtracted from the total current signal. Although the background, once subtracted, does not introduce a significant bias to the glucose measurement, it does significantly decrease the signal-to-noise ratio of the measurement in the hypoglycemic region. This increased noise increases the potential error in the glucose measurement in the hypoglycemic range. It is therefore important to determine the background current as accurately as possible. In some cases there is not enough time in the seven-minute cathodic cycle to consume H₂O₂ completely and the current at the end of this cycle is still decreasing. Therefore this measurement may not always provide the best estimation of the background. On the other hand, it was found that the current stabilizes earlier and more consistently in anodic cycles. Therefore, the baseline background is typically determined as the average of the last two current readings of the preceding anodic cycle.

After the background subtraction, the cathodic current signal is integrated to calculate the electrical charge (on the order of μC) liberated at the cathode, which is proportional to the total amount of glucose extracted through the skin. Integration has the added value that it compensates for variations in gel thickness and temperature, as these variables affect only the rate, not the extent of reaction. The integrated signal at the cathodal sensor for each half cycle are averaged as (C_{A}+C_{B})/2, a procedure that improves signal-to-noise ratio of the system.

Finally, the averaged charge signal is converted into a glucose measurement based on a patient's finger-stick calibration value (entered at the beginning of the monitoring period). From the calibration, a relationship between charge signal detected by the sensor and blood glucose is determined. This relationship is then used to determine glucose values based on biosensor signal measurements. The latter is achieved by utilizing a signal processing algorithm called Mixtures of Experts (MOE) (Kurnik, R. T., Sensors and Actuators B 60, 1 (1999); U.S. Pat. Nos. 6,180,416, 6,326,160, and 6,653,091). The MOE algorithm incorporates: integrated charge signal, calibration glucose value, charge signal at calibration, and time since calibration (i.e., elapsed time). It calculates each glucose reading as a weighted average of predictions obtained from three independent linear models (called Experts), which depend on the four inputs and a set of 30 optimized parameters. Equations to perform this data conversion have been developed, optimized, and validated on a large data set consisting of GlucoWatch biographer and reference BG readings from clinical trials on diabetic subjects. This data conversion algorithm is programmed into a dedicated microprocessor in the GlucoWatch biographer.

The GlucoWatch G2 biographer reduces warm-up time (from three to two hours), increases the number of readings per hour (up to six versus up to three), extends AutoSensor duration (use for 12 to 13 hours), and provides predictive low-alert alarms. The increase in the number of readings provided by the GlucoWatch G2 biographer is the result of a modified data processing algorithm that provides a series of moving average values based on the glucose-related signals from sensors A and B. The GlucoWatch G2 biographer uses the same AutoSensor as the first-generation GlucoWatch biographer.

The glucose readings provided by the GlucoWatch biographers lag the actual blood glucose by about 15-20 minutes. This lag is derived not only from the inherent measurement lag resulting from the time-averaging of glucose signals performed by the GlucoWatch biographers, but also from the physiological differences between the concentration of glucose in interstitial fluid (which is measured by the GlucoWatch biographers) and the instantaneous glucose concentration in blood (as typically measured via a finger prick). The measurement lag is 13.5 minutes. A GlucoWatch biographer glucose reading corresponds to the average glucose concentration in interstitial fluid during the two preceding 3-minute extraction periods (separated by the first 7-minute sensing period) and it is provided to the user after the second 7-minute sensing period, resulting in the 13.5 minute measurement lag. The additional physiological lag is estimated as about 5 minutes.

The GlucoWatch biographers perform a series of data integrity checks (see, e.g., U.S. Pat. No. 6,233,471 and 6,595,919) before computing each glucose value. The checks, called screens, selectively prevent certain glucose values from being reported to the user based on certain environmental, physiological, or technical conditions. The screens are based on four measurements taken during the course of wear: current (electrochemical signal), iontophoretic voltage, temperature, and skin surface conductance. Removed points are called skips. For example, if sweat is detected by an increased skin surface conductance, the glucose reading is skipped because the sweat could contain glucose, which could interfere with the glucose extracted from the skin during the iontophoretic period. Other skips are based on noise detected in the signal.

2.0.0 Modes of Carrying Out the Invention

Before describing the present invention in detail, it is to be understood that this invention is not limited to particular sampling methods, sensing systems, or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

2.1.0 General Overview of the Invention

The present invention relates generally to microprocessors, devices, and methods of monitoring of physiological analytes and detection of amounts or concentrations of such analytes. In one aspect, the present invention relates to improved selectivity of data screens. In another aspect, the present invention relates to compensation for fluctuations (e.g., sweat and/or temperature) that affect analyte measurement. The present invention provides for sweat and/or temperature detection that correlate more closely with changes in signals (e.g., amperometric or charge signals) related to analyte amount or concentration. The present invention provides for the establishment of more accurate sweat and/or temperature thresholds and new methods of compensation, such as correcting for the effects of sweat and rapidly changing temperature. When a subject is sweating, or when temperature is rapidly changing, the present invention (i) reduces the number of skipped or unusable readings experienced by the subject, and/or (ii) improves the sensitivity and/or specificity of the skips; further, the present invention provides methods for improving the accuracy of reported readings of analyte amount or concentration. In another aspect, the present invention relates to one or more passive collection reservoirs/sensing devices, present in conjunction with one or more active collection reservoirs/sensing devices, wherein the one or more passive collection reservoirs/sensing devices are used to provide information concerning sweat-related analyte and/or temperature changes (e.g., in the subject being monitored). In one embodiment, the present invention provides collection reservoir assemblies, collection reservoir/electrode assemblies, and AutoSensor assemblies, comprising one or more passive collection reservoir/electrode assemblies as well as one or more active collection reservoir/electrode assemblies. Such assemblies are typically consumable components of an analyte monitoring devices used to provide frequent measurement of the concentration or amount of one or more target analyte present in a biological system.

The present invention is useful in a variety of analyte monitoring devices that employ sampling methods that rely on methods that increase or enhance transdermal analyte flux, including, but not limited to, iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, and combinations thereof. These sampling methods are well known in the art, for example, iontophoresis (see, e.g., PCT International Publication Nos. WO 97/24059, WO 96/00110, and WO 97/10499; European Patent Application No. EP 0942 278; U.S. Pat. Nos. 5,771,890, 5,989,409, 5,735,273, 5,827,183, 5,954,685, 6,023,629, 6,298,254, 6,687,522, 5,362,307, 5,279,543, 5,730,714, 6,542,765, and 6,714,815), sonophoresis (see, e.g., Chuang H, et al., Diabetes Technology and Therapeutics, 6(1):21-30, 2004; U.S. Pat. Nos. 6,620,123, 6,491,657, 6,234,990, 5,636,632, and 6,190,315; PCT International Publication No. WO 91/12772; and Merino, G, et al, J Pharm Sci. 2003 June;92(6):1125-37), suction (see, e.g., U.S. Pat. No. 5,161,532), electroporation (see, e.g., U.S. Pat. No. 6,512,950, and 6,022,316), thermal poration (see, e.g., U.S. Pat. No. 5,885,211), use of microporation (see, e.g., U.S. Pat. No. 6,730,028, 6,508,758, and 6,142,939), use of microneedles (see, e.g., U.S. Pat. No. 6,743,211), use of microfine lances (see, e.g., U.S. Pat. No. 6,712,776), skin permeabilization (see, e.g., Ying Sun, Transdermal and Topical Drug Delivery Systems, Interpharm Press, Inc., 1997, pages 327-355), chemical permeation enhancers (see, e.g., U.S. Pat. No. 6,673,363), and use of laser devices (see, e.g., Gebhard S, et al., Diabetes Technology and Therapeutics, 5(2), 159-166, 2003; Jacques et al. (1978) J. Invest. Dermatology 88:88-93; PCT International Publication Nos. WO 99/44507, WO 99/44638, and WO 99/40848).

These methods may be affected by analyte collected via perspiration versus analyte collected by the sampling method. In one aspect, the present invention utilizes information obtained from a data stream, for example, frequently obtained analyte values (e.g., glucose related values), skin conductance, raw analyte-related signals (e.g., signal from an electrochemical biosensor), and/or temperature readings, generated by an analyte monitoring device (e.g., a GlucoWatch biographer system) to provide analyte measurement values having improved accuracy. The methods and devices described herein may be applied to single measurement values as well.

Further, perspiration may provide interference to measurements provided by an analyte monitoring device that measures glucose under the skin via RF-impedance. Transdermal, non-invasive, spectroscopic methods may also be affected by an extra glucose on the surface of the skin in sweat. These methods are also subject to variations in analyte measurements as a result of temperature fluctuations; the methods and devices of the present invention may be employed in conjunction with these technique as well. Accordingly, in one embodiment of the present invention, one or more passive collection reservoirs/sensing devices are present in conjunction with a spectroscopic sensing device.

The invention is described herein with reference to GlucoWatch biographer systems as an exemplary analyte monitoring system capable of providing frequent readings of analyte (e.g., glucose) amount or concentration for a subject. The GlucoWatch biographer system has been described above.

However, the microprocessors and methods of the present invention, as well as the one or more passive collection reservoirs/sensing devices described herein, can be used in numerous analyte monitoring devices to practice the present invention. Typically, the analyte monitoring device is used to monitor the level of an analyte (e.g., glucose) in a target system. Such an analyte monitoring device typically comprises a sensing device, which detects the amount or concentration of analyte (or a signal associated with the analyte amount or concentration) in samples provided use of a sampling method, and one or more microprocessors programmed to control operation of the sensing device, as well as to control the execution of a variety of analyses, algorithms, and/or methods, including the methods of the present invention. In a further embodiment, an analyte monitoring device comprises a sampling device, which provides one or more samples comprising the analyte, a sensing device, which detects the amount or concentration of analyte (or a signal associated with the analyte amount or concentration) in samples, and one or more microprocessors programmed to control operation of the sampling and/or sensing devices.

2.2.0 Compensation for the Effects of Sweat and Temperature Change and Improving Selectivity of Sweat and Temperature Screens

In one aspect, the present invention provides more precise methods and devices for improving the selectivity of data screens related to sweat and changing temperature relative to prior methods and devices. One major shortcoming of the standard sweat probe and thermistor methods for sweat and temperature transient compensation is that the signal level kinetics of such standard methods differ from the active systems due to the different physics involved with sweat accumulation and evaporation at a standard sweat probe (e.g., which measures skin conductivity) and different time constants of thermal conduction at a thermistor. These methods of sweat and temperature detection only loosely correlate with changes in signal. Therefore, tight thresholds must be set or degraded accuracy in the glucose readings results. The present invention embodies methods and devices for temperature and sweat detection that correlate more closely with changes in signal. This enables the setting of more accurate thresholds and the application of new screening and/or compensation methods, including correcting for the effects of sweat and rapidly changing temperature. When a subject is sweating, or when temperature is rapidly changing, the present invention reduces the number of skipped readings experienced by the subject and can be used to improve accuracy for reported analyte measurement values.

2.2.1 Methods for Compensation Relating to Analyte in Sweat

Sweat is known to contain a number of analytes of interest, for example, glucose. Perspiration can affect the function of transdermal analyte monitoring devices and/or the accuracy of analyte-related measurement values obtained using transdermal analyte monitoring devices. For example, during perspiration (employing the GlucoWatch biographer as an exemplary analyte monitoring device) extra glucose, i.e., glucose that was not actively extracted by the analyte monitoring system, is collected by the hydrogel pads from the skin beneath them and is sufficient to cause significant error in the glucose measurements obtained during periods of perspiration. The only physiological condition that has been shown to disrupt calibration of the GlucoWatch biographers is perspiration. In the GlucoWatch biographer G2, the presence of perspiration is detected by skin conductivity probes mounted on the underside of the device. When the perspiration reaches a certain threshold the GlucoWatch biographer skips the glucose reading associated with the perspiration event, i.e., the reading is not displayed to the user. The threshold (i.e., degree of perspiration) was determined during the developmental clinical trials by investigating the average error associated with GlucoWatch biographer readings with differing skin conductivity readings. The threshold was set at a level to exclude points with unacceptably high average error.

Before a GlucoWatch G2 biographer reading is presented to the user, a number of parameters of the biosensor signals and the GlucoWatch G2 biographer's operation are checked against predetermined criteria to ensure data integrity (see, e.g., U.S. Pat. Nos. 6,233,471 and 6,595,919). These parameters include low or rapidly changing temperature, the presence of excess perspiration, excess noise in the raw signal, or sensor connection faults. If any of these parameters is detected, the glucose reading is skipped to ensure the accuracy of the glucose measurements. If the data passes these checks, the biosensor signals as well as a calibration factor determined from a fingerstick blood glucose measurement taken two hours after start are input to an algorithm to calculate a glucose reading. Subsequent readings are presented to the user up to every 10 minutes for up to 13 hours.

As described above, one of the conditions that causes the GlucoWatch G2 biographer to skip readings is perspiration. It would be beneficial to the user to be able to view glucose readings that are presently skipped. There are a number of cases where the ability to detect glucose during perspiration would be especially useful to the diabetic. First, and most importantly, perspiration is often a symptom of hypoglycemia. Although the user is alerted that a potential glucose reading is being skipped because of perspiration, and can review previous Biographer readings for evidence of decrease glucose levels, obtaining actual glucose readings during this time would increase the usefulness of the device to the user, and increase the sensitivity of the hypoglycemia alert feature. Second, monitoring glucose levels during exercise is important for diabetics to prevent exercise-induced hypoglycemia. Additionally, use of the Biographer by persons living in hot, humid climates, or by overweight, heavily perspiring persons is hindered by the effect of perspiration on the glucose measurements.

Although this discussion is with reference to the GlucoWatch biographer analyte monitoring devices, the effect of perspiration is not limited to the reverse-iontophoresis extraction method used in the GlucoWatch biographer. Other transdermal extraction methods (including, but not limited to, sonophoresis (ultrasound-induced skin permeabilization (Kost, J., et al., Nat. Med. 6:347-350, 2000)), microneedles (Smart, W. H., et al., Diab. Tech. Ther. 2(4):549-559, 2000), laser poration (Gebhart, S., et al., Diab. Tech. Ther. 5:159-168, 2003)) are affected by the presence of glucose delivered by perspiration, rather than glucose delivered by the specific extraction method. These techniques, which are used for transdermal glucose monitoring, require calibration in a manner analogous to that used in the GlucoWatch biographer; this calibration will be affected by the extraneous glucose delivered through sweat. Calibration methods employed may be single or multiple-point calibrations. Calibration methods may take into account previously determined calibration values. Perspiration may also affect a “non-invasive” glucose monitor (in development, Caduff, A., et al., American Diabetes Association 62^{nd} Scientific Sessions, San Francisco, June 14-18, 2002, Diabetes 51:(Supp.2), A119, 2002) utilizing an RF-impendence method. It is possible that spectroscopic methods, such as near-infrared methods, are affected by the presence of extraneous glucose in sweat on the skin surface, especially those near-infrared systems being developed for continuous monitoring. Thus, the methods and devices described herein for correction of sweat-induced errors are generalizable to many transdermal and non-invasive glucose monitoring methods.

In the GlucoWatch G2 biographer, approximately 2-3% of all readings are skipped because of perspiration. However, these skipped readings are not randomly distributed and tend to occur in clusters. i.e., several readings are skipped sequentially during a period of perspiration.

The readings from the skin conductivity detector of the GlucoWatch biographers indicate the presence of perspiration as well as the degree of perspiration. Skin conductivity readings range from 0 to 10 μS. Readings above 1.0 μS, in the GlucoWatch biographer and the GlucoWatch biographer G2, presently result in skipped readings. Data obtained during the optimization of this threshold showed that the number of glucose measurements falling into the undesirable C, D and E regions of the Clarke Error Grid was twice as high for readings that were skipped than the readings that were presented to the user.

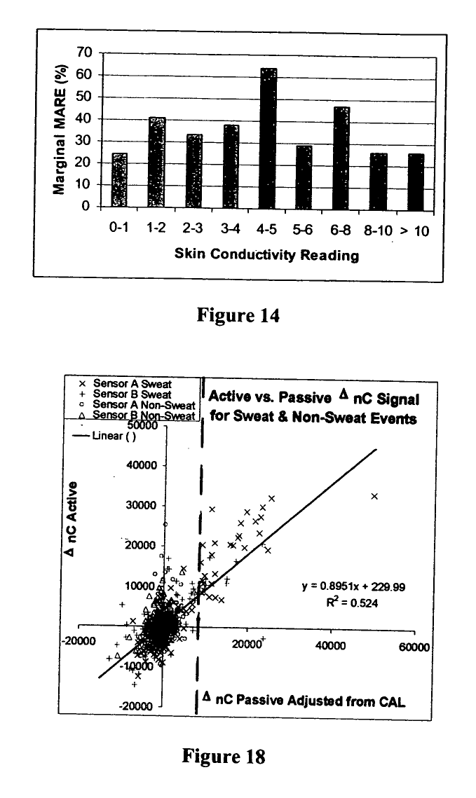

FIG. 14 presents data showing Mean Absolute Relative Error (MARE) of GlucoWatch biographer glucose readings calculated when the GlucoWatch biographer detected variable amounts of sweat. MARE was calculated relative to blood glucose measurements measured via fingerstick method at the different skin conductivity values. From these results it can be seen that error of a glucose measurement during perspiration (higher skin conductivity) tends to increase relative to measurements taken during periods of non-perspiration (lower skin conductivity). MARE for the points not skipped (skin conductivity reading 0-1) was 24.4%; MARE for all sweat reading above this had higher errors; although a linear trend is not seen. This simple analysis takes into account skin conductivity alone.

In one aspect, the present invention relates to methods to correct the glucose readings during perspiration, rather than merely skipping the readings. Correcting GlucoWatch biographer readings during periods of perspiration, rather than merely skipping the readings, provides improved usefulness to persons with diabetes using the GlucoWatch biographer, allowing better management of their glycemic levels.

In a first method, readings from the skin conductivity detector, as well as the characteristics of the biosensor signals themselves are used to detect periods of perspiration and to correct the glucose biosensor signal for the glucose sampled through perspiration, rather than glucose extracted by iontophoresis. This method may be incorporated into the GlucoWatch biographer via firmware and software changes (e.g., one or more microprocessors of the GlucoWatch biographer may be programmed to control operation of the GlucoWatch biographer and execute algorithms associated with the method).

In one aspect of the present invention, an analyte level can be used as an input to a sweat correction algorithm that may be employed in a variety of analyte monitoring devices. Other measured parameters may be added to the correction algorithm as well. In one embodiment, a biosensor reading during periods of sweating is corrected using a number of parameters collected by an analyte monitoring device (e.g., skin conductivity, temperature, biosensor integral, biosensor baseline background and anodic biosensor integral and background). Ideally, one or several of these readings are directly related to the amount of extra analyte delivered to the biosensor by sweat. For example, a sweat probe reading, which is a measure of skin conductivity, may be used as a correction factor to correct the analyte measurement value from an extracted sample. When there is a direct relationship between the skin conductivity reading and the degree of perspiration, then the amount of analyte delivered to the biosensor associated with the extracted sample may be proportional to the degree of perspiration. Thus, a proportionality constant is established between the skin conductivity reading and the amount of error caused by the analyte in perspiration. The degree of error is also proportional to the analyte amount or concentration in the subject being monitored at the time, as sweat will have a higher analyte concentration at higher analyte levels in the subject. In addition to proportionality constants, other linear or non-linear functions can be employed that relate skin conductivity to the amount of error caused by the analyte in perspiration. The exact function used for the correction may have a number of forms empirically determined following the teachings of the present disclosure. The above scheme assumes that the biosensor signal (either raw or integrated) is corrected; but another possibility is to correct a final analyte measurement value before display.

This aspect of the present invention is exemplified herein below with the reference to the GlucoWatch G2 biographer and electrochemical analyte detection of analyte (in this case, glucose). It is already known that a number of parameters collected by the GlucoWatch G2 biographer are affected by the presence of sweat (e.g., skin conductivity, temperature, biosensor integral, biosensor baseline background and anodic biosensor integral and background). Ideally, one or several of these readings are directly proportional to the amount of extra glucose delivered to the biosensor hydrogel by sweat. For example, the sweat probe reading, which is a measure of skin conductivity, may be used as in a correction factor to correct the cathodic glucose biosensor reading. When there is a direct relationship between the skin conductivity reading and the degree of perspiration then the amount of glucose delivered to the biosensor hydrogel may be proportional to the degree of perspiration. Thus, a proportionality constant is established between the skin conductivity reading and the amount of error caused by the glucose in perspiration. The degree of error is also be proportional to the blood glucose at the time as sweat would have a higher glucose concentration at higher blood glucose levels, leading to, for example, the following correction equation:

Biosensor (corrected)=Biosensor (measured)*SC*K*BG, where Biosensor (measured) is the biosensor measurement that needs to be corrected, SC is the skin conductivity reading, K is a proportionality constant, and BG is the blood glucose (which could be approximated by the last GlucoWatch biographer reading unaffected by sweat).

In an alternate embodiment, wherein linearity is not assumed:
Biosensor (corrected)=f (Biosensor(measured), SC, BG)

The exact function used for the correction could have a number of forms empirically determined following the teachings of the present disclosure. The above scheme assumes that the biosensor signal (either raw or integrated) is corrected; but another possibility is to correct the final glucose reading before display. In yet another embodiment the sweat probe reading may be included as an input parameter to MOE or other optimized algorithm. To do this, the algorithm is optimized using a data set that includes readings that occur during sweating.

The GlucoWatch G2 biographer glucose measurement is taken from the biosensor that is at the iontophoretic cathode as glucose is collected mainly into this hydrogel pad. The other pad, at the iontophoretic anode, normally collects mainly anodic interfering species, such as ascorbic acid and uric acid. The biosensor at this anodic pad is activated during the biosensing period, but the signal from this electrode is not used in the glucose measurement. During periods of no sweating, the anodic biosensor signal contains components mainly from anionic interfering species (e.g., ascorbic acid and uric acid) and only a small amount of glucose. The anodic signal does not change greatly from cycle to cycle. During periods of sweating, however, glucose is delivered to the iontophoretic anode hydrogel by the sweat, which results in a signal from this glucose, as well as the hydrogel at the iontophoretic cathode. Parameters of the anodic biosensor signal may be used to subtract off the signal from the glucose delivered via sweat from the glucose biosensor signal. For example, if a signal from an anodic cycle before the sweating occurs is subtracted from a signal from an anodic cycle during sweating, the difference is the signal from the glucose delivered via sweat. This difference can then be subtracted from the cathodic biosensor signal to “correct” the signal for the additional glucose. A proportionality constant that takes into account the relative average signals from the two sensors (similar to A/B and B/A ratios used in extrapolation and interpolation, see, e.g., PCT International Publication No. WO/03/000127) may be used in this case as well to take into account the different biosensor sensitivities, skin sites, etc. during the subtraction process. Subtraction can be done either point by point, or as integrals. The corrected biosensor signal may then be an input parameter for the MOE algorithm as usual.

2.2.2 Methods and Devices for Compensation Related to Temperature

In another aspect, the present invention relates to a reference collection reservoir used to obtain information about temperature fluctuations in the subject being monitored using an analyte monitoring device. The reference collection reservoir is typically isolated from the active collection reservoirs (e.g., the reference collection reservoir is electrically isolated from active collection reservoirs) and there is no direct contact between this collection reservoir and the skin surface of the subject being monitored. In some embodiments of the present invention, the reference collection reservoir corresponds to a passive collection reservoir/sensing device assembly, wherein a mask layer prevents movement of analyte into the collection reservoir from the skin surface in which the reservoir is in operative contact. In some embodiments of the present invention, a thermistor may be in operative contact with the reference collection reservoir/sensing device. Alternatively, a thermistor may be in close proximity to the sensing device that is in operative contact with the reference collection reservoir, or be in thermal equilibrium with the sensing device, for example, a thermistor may be in close proximity to an electrode assembly comprising a sensing electrode, which is in contact with a hydrogel.

In one embodiment, the present invention comprises a reference collection reservoir/sensing device, circuitry and software to obtain information about skin temperature variations/fluctuations and transient background signals. In embodiments wherein the sensing device comprises one or more sensing electrodes, the reference collection reservoir is electrically isolated from active collection reservoirs associated with sensing electrodes. Typically there is no direct contact between the reference collection reservoir and the skin. Isolation from the skin is typically effected by placing the reference collection reservoir behind a mask layer. The collection reservoir may be interrogated by the sensing device of the analyte monitoring device in the same way as active collection reservoirs (e.g., a three electrode electrochemical cell preferably using the same counter and working electrode materials; but different geometries may be used if the reference collection reservoir is, for example, smaller than active collection reservoirs).

For the purpose of illustration, following is a description with reference to the GlucoWatch G2 biographer as an exemplary analyte monitoring device. Temperature correction is already performed in the GlucoWatch G2 biographer; but it is done with a thermistor internal to the device. The thermistor is a few millimeters from the skin surface. The location of the thermistor affects temperature dynamics and, accordingly, readings taken at the thermistor may not always accurately reflect what is happening at the skin surface. In contrast, the reference collection reservoir of the present invention is only separated from the skin surface by, for example, a thin mask material.

Using the information gathered by the reference collection reservoir/sensing device, a blank transient signal (i.e., signal obtained in the absence of analyte) may be used, for example, for baseline subtraction of integrated signal. This blank transient signal is a function, for example, of properties of the sensing device in contact with the reference collection reservoir (e.g., electrode components that are in contact with the reference collection reservoir, electrode/gel capacitance, and other electrochemical phenomena). Correct compensation for such blank transient signal can be used to improve performance of analyte monitoring devices, for example, the GlucoWatch biographers.

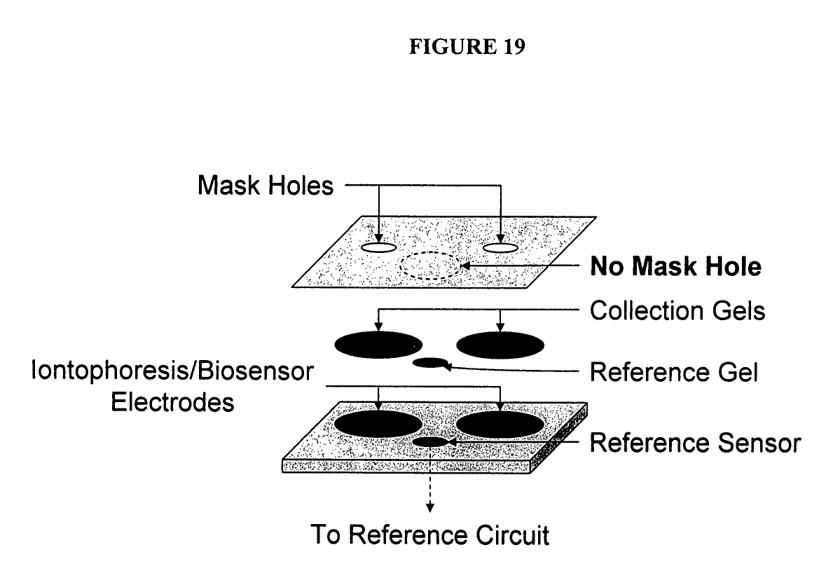

FIG. 19 presents an example of a reference collection reservoir (“reference gel” in the figure) of the present invention. In the figure, the reference gel cannot contact the skin surface because the mask layer intervenes. The mask layer defines openings that allow the passing of analyte into the active collection reservoirs (“collection gels” in the figure). The active collection reservoirs can be placed in operative contact with the iontophoretic/biosensor electrodes. The reference collection reservoir can be placed in operative contact with the appropriate electrodes (“reference sensor” in this figure).

The reference collection reservoir/sensing device may be coupled with appropriate circuitry to obtain signal and algorithms for analysis of signal (e.g., “reference circuit” in the figure). The signal or analyzed signal may be used as input into further algorithms.

Although the invention is exemplified with reference to the GlucoWatch biographer, the invention may be applied to other analyte monitoring devices by one of ordinary skill in the art in view of the teachings of the present specification.

2.2.3 Further Methods of and Devices for Sweat and Temperature Screening and Compensation as Well as Possible Principles of Operation

The present invention generally relates to methods and devices for improving, relative to previously used methods and devices, the selectivity of data screens related to sweat and changing temperature. The present invention also includes microprocessors comprising programming to control such methods that may be components of analyte monitoring devices. Further, the present invention generally relates to methods of compensation for fluctuations (e.g., sweat and/or temperature) that affect analyte measurements. Such methods and devices of the present invention may be employed in a variety of analyte monitoring devices, including, but not limited to, analyte monitoring devices that employ methods to enhance transdermal or transmucosal transport of analyte, such as iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, and combinations thereof. These sampling methods are well known in the art, for example, iontophoresis (see, e.g., PCT International Publication Nos. WO 97/24059, WO 96/00110, and WO 97/10499; European Patent Application No. EP 0942 278; U.S. Pat. Nos. 5,771,890, 5,989,409, 5,735,273, 5,827,183, 5,954,685, 6,023,629, 6,298,254, 6,687,522, 5,362,307, 5,279,543, 5,730,714, 6,542,765, and 6,714,815), sonophoresis (see, e.g., Chuang H, et al., Diabetes Technology and Therapeutics, 6(1):21-30, 2004; U.S. Pat. Nos. 6,620,123, 6,491,657, 6,234,990, 5,636,632, and 6,190,315; PCT International Publication No. WO 91/12772; and Merino, G, et al, J Pharm Sci. 2003 June;92(6):1125-37), suction (see, e.g., U.S. Pat. No. 5,161,532), electroporation (see, e.g., U.S. Pat. No. 6,512,950, and 6,022,316), thermal poration (see, e.g., U.S. Pat. No. 5,885,211), use of microporation (see, e.g., U.S. Pat. No. 6,730,028, 6,508,758, and 6,142,939), use of microneedles (see, e.g., U.S. Pat. No. 6,743,211), use of microfine lances (see, e.g., U.S. Pat. No. 6,712,776), skin permeabilization (see, e.g., Ying Sun, Transdermal and Topical Drug Delivery Systems, Interpharm Press, Inc., 1997, pages 327-355), chemical permeation enhancers (see, e.g., U.S. Pat. No. 6,673,363), and use of laser devices (see, e.g., Gebhard S, et al., Diabetes Technology and Therapeutics, 5(2), 159-166, 2003; Jacques et al. (1978) J. Invest. Dermatology 88:88-93; PCT International Publication Nos. WO 99/44507, WO 99/44638, and WO 99/40848).

Microprocessors, methods and devices of the present invention provide for detection of temperature- and sweat-induced signals that correlate more closely with changes in signal than standard sweat probe and thermistor methods of sweat and temperature detection.

These methods and devices typically related to the use of a passive collection reservoir associated with a sensing device that provides signals related to analyte measurement values providing an amount or concentration of analyte in a transdermally/transmucosally extracted sample. The passive collection reservoir/sensing device collects analyte passively, which is a sensitive indicator of analyte collected via sweat. The signal from this passive collection reservoir/sensing device may be used to subtract out the signal arising from analyte in sweat from the signal in the sample obtained by transdermal extraction. One advantage of this method, versus subtracting the analyte from sweat using the anodic biosensor signal as the passive biosensor (as described above), is that the passive collection reservoir/sensing device typically provides a small signal during non-sweat periods in contrast to the anodic biosensor whose signal would contain components arising from the interfering species compounds normally collected at the anode.

Alternately or in addition, the signal from a passive collection reservoir/sensing device may be used, for example, to set thresholds related to data screening. When a subject is sweating, or when temperature is rapidly changing, the present invention reduces the number of skipped readings experienced by the subject and can be used to improve accuracy for reported analyte measurement values obtained through use of an analyte monitoring device. Accordingly, the signal from this passive collection reservoir/sensing device may be used to improve the selectivity of data screens based on sweat and/or temperature fluctuations.

In one embodiment of this aspect of the present invention, for example for use in a GlucoWatch biographer monitoring devices, the passive collection reservoir/sensing device comprises a hydrogel (e.g., comprising the enzyme glucose oxidase) that can be placed in operative contact with a sensing device (e.g., comprising a sensing electrode that provides electrochemical signal).

Though not wishing to be bound by any particular theory or hypothesis concerning methods of operation, the following discussion is presented to facilitate understanding of some aspects of the present invention.

Experiments performed in support of the present invention indicated that the analyte-related signal obtained from a passive collection reservoir/sensing device was a good predictor of sweat and/or temperature-transient induced signals obtained from at the active electrodes. In one embodiment, this may be achieved by designing the passive system to have signal level kinetics as similar to the active systems, being used to obtain analyte measurement values, as possible (i.e., the passive system and the active system have substantially the same physical characteristics). For example, when the active detection system is electrochemical, some key design variables typically include using similar materials, similar thickness dimensions, similar methods of fabrication, similar electrical excitation, and similar electrical sensing, for the passive system as for the active systems. This approach addresses a major shortcoming of the standard sweat probe and thermistor methods of sweat and temperature transient compensation, in that, the signal level kinetics of such standard methods differ from the active systems due to the different physics involved with sweat accumulation and evaporation at the sweat probes and different time constants of thermal conduction at the thermistor.

Some attributes of the present invention can be described employing exemplary compensation and/or screening methods/algorithms that employ a number of parameters. The screening methods/algorithms typically provide improved data selectivity relative to previously used methods.

Methods of the present invention for providing selectivity for data screens and compensating for fluctuations (e.g., sweat and/or temperature) that affect analyte measurements are applicable to a variety of transdermal analyte monitoring devices and data obtained there from. The microprocessors, devices, and methods of the present invention are applicable to a wide variety of analyte monitoring devices including, but not limited to, those employing the following transdermal or transmucosal extraction methods: iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, and combinations thereof. Some aspects of the present invention are exemplified herein with the reference to the GlucoWatch biographer and electrochemical detection of analyte (in this case glucose is the exemplified analyte).

As an illustration of the present invention, the following parameters affecting measurement values, for example, as determined by the GlucoWatch biographers are used. The GlucoWatch G2 biographer signal (integrated electrical current over time) is used as an indicator of blood glucose through the use of a Mixture of Experts (MOE) algorithm (see, e.g., U.S. Pat, Nos. 6,180,416, 6,326,160, and 6,653,091). In general terms, the algorithm predicts blood glucose (BG) as a function of: input signal (integrated electrical current) for the current biosensing period (Q), the measured blood glucose at the time of calibration (BGcal), the input signal (integrated electrical current) at the time of calibration (Qcal), and the elapsed time (ET) since starting use of the device. This relationship is represented by the following equation:
BG=f(Q, BGcal, Qcal, ET).

This relationship can be applied to other analytes as well by those of skill in the art in view of the teachings of the present specification.

The following parameters are used in the exemplary compensation/screening algorithms described herein below:

BG=predicted blood glucose value;

f( . . . )=function of;

Q=input signal (integrated electrical current typically expressed in units of nano-coulombs (nC), with the baseline current subtracted from the measured current);

BGcal=measured blood glucose value at the time of calibration;

Qcal=input signal at the time of calibration;

ET=elapsed time from the start of using the device;

Qa=active signal (integrated electrical current at the active electrodes typically expressed in units of nano-coulombs, with the baseline current subtracted from the measured current);

Qag=active glucose signal (portion of the integrated electrical current at the active electrodes that is due to iontophoresis induced glucose flux through the skin);

Qas=active sweat signal (portion of the integrated electrical current at the active electrodes that is due to sweat, which includes glucose in the sweat and any species that react directly at the working electrodes);

Qat=active temperature transient signal (portion of the integrated electrical current at the active electrodes that is due to temperature transients during the biosense period);

Qacal=active signal at time of calibration;

Qabl=active baseline signal;

Qpp=passive signal from the passive extract and biosensor background (portion of the integrated electrical current at the passive electrodes that exists due to background currents inherent in the collection reservoir/electrodes and passive diffusion of glucose and/or electrochemically active species into the collection reservoir/hydrogel);

Qp=passive signal (integrated electrical current at the passive electrodes typically expressed in units of nano-coulombs, with the baseline current subtracted from the measured current);

Qps=passive sweat signal (portion of the integrated electrical current at the passive electrodes that is due to sweat, which includes glucose in the sweat and any species that react directly at the working electrodes);

Qpt=passive temperature transient signal (portion of the integrated electrical current at the passive electrodes that is due to temperature transients during the biosensing period);

Qpcal=passive signal at time of calibration;

Qpbl=passive baseline signal;

k=proportionality factor (typically a fractional value between 0 and 1, but may include the values of 0 or 1);

k1=proportionality factor number 1;

k2=proportionality factor number 2;

Qpthresh.=threshold value for the passive signal, above which, prediction of the blood glucose value is skipped;

Qpthresh1=lower threshold value for the passive signal, below which, prediction of the blood glucose value is skipped;

Qpthresh2=upper threshold value for the passive signal, above which, prediction of the blood glucose value is skipped; and

Qpcalthresh=threshold value for passive signal, above which, calibration by the user is not be accepted.

The integrated current (Qa) at the two active collection reservoir electrode systems can be modeled as a combination of actively extracted glucose signal (Qag), sweat signal at these active electrodes (Qas), and temperature transient signal at these active electrodes (Qat). (In the case of the GlucoWatch biographers the two physical sensing electrodes are not used simultaneously to measure glucose; rather, they are used alternatingly. The glucose-related signals from these two sensing electrodes can be used singly or in a number of combinations (see, e.g., PCT International Publication No. WO 03/000127)).
Qa=Qag+Qas+Qat

The integrated current at the passive collection reservoir/sensing system (e.g., passive collection reservoir/sensing electrode) (Qp) can be modeled as a combination of sweat signal at this passive electrode (Qps), and temperature transient signal at this passive electrode (Qpt).
Qp=Qps+Qpt+Qpp

The present invention teaches that the integrated current at the passive third collection reservoir electrode system (Qp) is a good predictor of the sweat (Qas) and/or temperature transient induced signal (Qat) at the active electrodes.
Qas+Qat=f(Qp)

One functional relationship is the case where the passive signal (Qp) matches the sweat (Qas), temperature transient (Qat), and Qpp=0.
Qp=Qps+Qpt=Qas+Qat

In this simple case, the signal input to the algorithm for calculating blood glucose (Q,) could be the difference between the active electrode signal (Qa) and the passive electrode signal (Qp), that is the active glucose signal (Qag).
Q=Qa−Qp=Qag

In a different case, the passive signal may be used as an indicator of when the active signal should be ignored, and the predicted glucose value skipped, for example:

if Qp is less than or equal to Qpthresh, then Q=Qa,

if Qp is greater than Qpthresh, then Q=skip reading.

These simple relationships do not necessarily take full advantage of the passive electrode signal. In a more general case, it may be useful to (i) use a proportionality factor, (ii) take special account of the passive signal at calibration, (iii) account for elapsed time, and/or (iv) include the level of the active signal, active signal at calibration, baseline of the active signal, and baseline of the passive signal. The following equation indicates a functional relationship of the signal input to the algorithm (Q), to particular attributes of the raw signal from the sensors. Typical functional relationships, shown as examples, are linear relationships. Alternatively, the relationships could include logarithmic decay type functions.
Q=Qa−f(Qp, Qpcal, ET, Qa, Qacal, Qabl, Qpbl).

Examples of possible compensation/screening algorithms include, but are not limited to, the following:
(A) Q=Qa−k Qp;
(B) Q=Qa−k Qp Qacal/Qpcal;
(C) Q=Qa−k(Qp−Qpcal);
(D) Q=Qa+k1Qabl−k2 Qpbl;
(E) Q=f(Qa, ET)−k Qp, where f(Qa, ET) is the active signal level after compensating for the effects of signal decay;
(F) Q=f(Qa, ET)−k Qp Qacal/Qpcal, where f(Qa, ET) is the active signal level after compensating for the effects of signal decay;
(G) Q=f(Qa, ET)−k(Qp−Qpcal), where f(Qa, ET) is the active signal level after compensating for the effects of signal decay;
(H) Q=f(Qa, ET)+k1 Qabl−k2 Qpbl, where f(Qa, ET) is the active signal level after compensating for the effects of signal decay;
(I) Q=Qa, when |Qp−Qpcal| is less than or equal to Qpthresh;
(J) Q=skip reading, when |Qp−Qpcal| is greater than Qpthresh;
(K) Q=Qa, when Qp/Qpcal is greater than or equal to Qpthresh1 and Qp/Qpcal is less than or equal to Qpthresh2; and/or
(L) Q=skip reading, when Qp/Qpcal is less than Qpthresh1 or Qp/Qpcal greater than Qpthresh2.

Proportionality factors may be affected by the following: (i) the ratio of skin area exposed to passive electrode to skin area exposed to each active electrode; (ii) the ratio of electrode area for passive electrode versus active electrode (this effect is due to the fact that background scales with electrode area); and (iii) the ratio of sweat flux for active electrode to passive electrode (e.g., iontophoresis may cause a different sweat flux as compared to an area of skin that has no iontophoresis). Proportionality factors may be empirically determined based on the teachings of the present specification. While not explicitly illustrated with an example, logarithmic decay proportionalities may also be employed.

In examples B, C, F, G, I, and J, one possible proviso is to require the passive signal at the time of calibration to be below a pre-determined threshold value, for example:

Calibrate only if Qp is less than or equal to Qpcalthresh.

Yet another embodiment of use of the passive signal is to use it as an input to the blood glucose prediction algorithm (e.g., Mixtures of Experts), for example:
BG=f(Qa, Qp, BGcal, Qacal, Qpcal, ET).

In a further embodiment of the present invention, the passive signal may be used for sweat compensation only, and a thermistor reading of temperature changes may be used for temperature transient compensation.

The efficacy of the passive collection reservoir/sensing device method of the present invention was tested as described in Example 1. Pairs of GlucoWatch G2 biographers were applied to the same subject (three pairs to each subject). Subjects were fasted to obtain constant blood glucose levels. One GlucoWatch biographer in each pair functioned normally using active iontophoretic extraction of glucose. The other GlucoWatch biographer in the pair was specially programmed to operate in a passive mode where no iontophoretic extraction of glucose took place. This approach provided a means to compare active and passive signals.

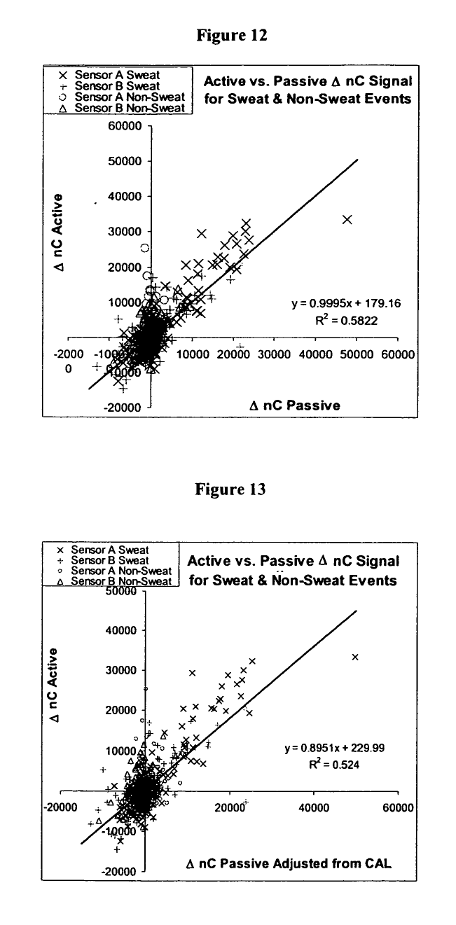

The results of the experiment demonstrated that while blood glucose values were substantially constant during sweat and non-sweat events, the signal varied significantly. There was a very significant correlation (FIG. 12) between the active and passive sweat and temperature related signals (i.e., Qas+Qat plotted versus Qps+Qpt), particularly considering that the GlucoWatch biographers were not in close proximity to each other. FIG. 13 shows a similar plot with the passive signal adjusted from its calibration value (Qp−Qpcal) as the estimate of the passive sweat and temperature related signal.

Because many biosensor readings obtained during sweat events show little change, the sweat probes alone may not be completely accurate in detecting sweat events that affect biosensor measurements. The methods presented herein provide more accurate detection of sweat events that affect analyte measurement.

The sweat-point data presented in Example 1 demonstrated that a passive collection reservoir/sensing device can measure signal that occurred due to temperature and/or sweat perturbations. FIG. 12 shows a graph representing the difference in integrated biosensor signal from the biosensor at the iontophoretic cathode under sweating conditions minus the integrated biosensor signal under non-sweating conditions (i.e., Qa−Qag, which is equal to Qas+Qat) versus the difference in integrated biosensor signal from the passive biosensor under sweating conditions minus the integrated biosensor signal under non-sweating conditions (i.e., Qp−Qpp, which is equal to Qps+Qpt). There was a good correlation between the signal perturbations for the two different sensors. This data suggests that correcting the iontophoretic glucose signal for sweat-/temperature-induced errors is possible using the glucose signal from a passive (i.e., non-iontophoretic) collection reservoir/sensing electrode.

Another embodiment of this aspect of the invention includes a biosensor (e.g., a collection reservoir/sensing electrode) that is not in chemical contact with the skin; rather, merely in physical contact with it (e.g., skin in contact with a mask layer, mask layer covering the collection reservoir, i.e., the mask does not define an opening to expose the collection reservoir). Such a biosensor would not detect analyte (e.g., glucose), but may serve as a source for a reference signal that could be subtracted from the analyte signal at an active biosensor to correct for temperature fluctuations during the biosensing cycle.

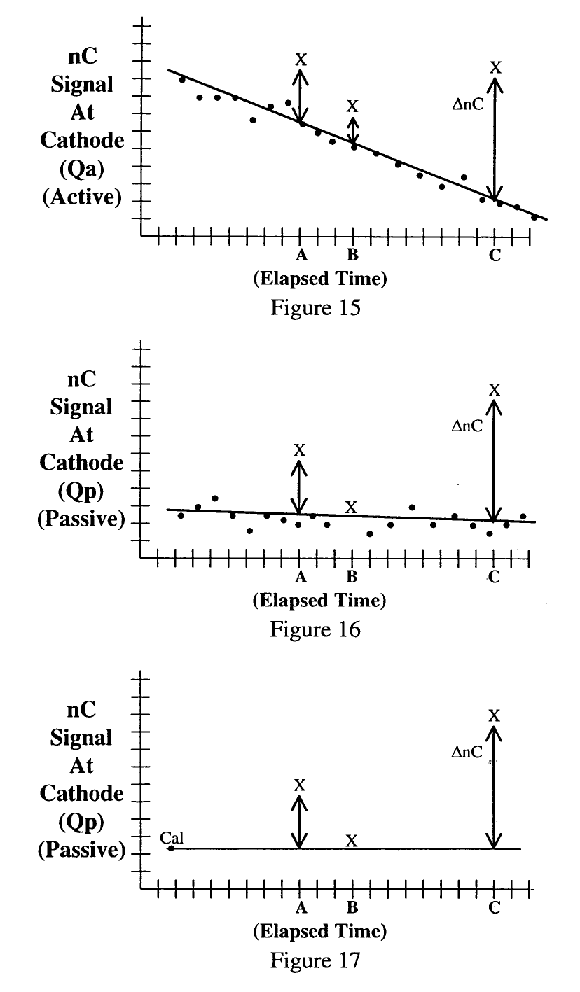

The results presented in Example 1 supported the use of a passive collection reservoir/sensing electrode not only for selective screening of data associated with sweat events, but for correction of data associated with sweat events as well. For example, data points at elapsed times (ETs) A, B, and C, presented in FIG. 15 and FIG. 17, illustrate applications of several aspects of the present invention. At time point A, Q=Qa−Qp, that is, Qp is used as a correction for the contribution to input signal (Q) related to sweat (Qps) and temperature factors (Qpt). At time point B, the contribution of Qp to overall signal, during a period of sweating, is negligible. Accordingly, a data screen can be applied in this that allows Q=Qa without any further correction. This is an example of improved data selectivity. A different example of improved data selectivity is illustrated at time point C, wherein Qp≈Qa. In this situation a data screen may be applied to skip the measurement value at this time point due to an overwhelming contribution to signal by the sweat related signal. In addition, a threshold may be set (e.g., Qpthresh) based on analysis of the data, wherein measurement values associated with a passive signal above a certain value are skipped. This threshold can then be used as a data screen. An example of one such Qpthresh is shown by the vertical dotted line in FIG. 18, wherein if Qp is less than or equal to Qpthresh, then Q=Qa; if Qp is greater than Qpthresh, then Q=skip reading.

Accordingly, the present invention relates to one or more microprocessors comprising programming to control performance of the methods of the present invention, as well as devices comprising such microprocessors or that perform such methods. In one embodiment, the one or more microprocessors provide a first signal related to analyte amount or concentration in a subject from a first sample comprising an analyte, wherein the first sample is obtained by use of a method that enhances transport of the analyte across a skin or mucosal surface of the subject. Further, the one or more microprocessors provide a second signal related to analyte amount or concentration from a second sample comprising the analyte, wherein the second sample is obtained substantially without use of a method that enhances transport of the analyte across the skin or mucosal surface of the subject, and the first signal and the second signal are obtained for substantially a same time period. The one or more microprocessors then qualify the first signal, for example, by a method selected from the group consisting of (i) screening the first signal based on the second signal; (ii) applying a correction algorithm to the first signal, wherein the first signal is adjusted by use of the second signal; and (iii) combinations thereof.

In a further embodiment, the qualifying comprises screening the first signal based on the second signal. For example, the screening comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, the screening may compare a signal trend to a predetermined set of signal trends, and the skipping or accepting may be based on matches between the signal trend and one or more predetermined set of signal trends.

In another embodiment, the qualifying further comprises obtaining a skin conductance value for substantially the same time period as the first and second signals, comparing the skin conductance value to a predetermined skin conductance threshold value, and if the skin conductance value equals or exceeds the skin conductance threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high signal threshold value and the low signal threshold value. Alternatively or in addition, a trend of skin conductance values may be compared to a set of predetermined trends of skin conductance values and a decision to further screen the signal may be based on matches between the skin conductance trend and one or more predetermined set of skin conductance trends. Further, subsequent screening may compare a signal trend to a predetermined set of signal trends, and the skipping or accepting may be based on matches between the signal trend and one or more predetermined set of signal trends.

In yet another embodiment, the qualifying further comprises obtaining a temperature value for substantially the same time period as the first and second signals, comparing the temperature value to a predetermined high and/or low temperature threshold value, and if the temperature value is above the high temperature threshold value or below the low temperature threshold value, then the first signal is screened based on the second signal. An exemplary screening method comprises (a) comparing the second signal to a predetermined high and/or low signal threshold value, (b) skipping an analyte measurement value associated with the first signal if the second signal is above the high signal threshold value or below the low signal threshold value, and (c) accepting the first signal for determination of an associated analyte measurement value if the second signal is between the high threshold value and the low threshold value. Alternatively or in addition, a trend of temperature values may be compared to a set of predetermined trends of temperature values and a decision to further screen the signal may be based on matches between the temperature trend and one or more predetermined set of temperature trends. Further, subsequent screening may compare a signal trend to a predetermined set of signal trends, and the skipping or accepting may be based on matches between the signal trend and one or more predetermined set of signal trends.

In additional embodiments, the qualifying comprises use of both of the above-described analyses for skin temperature values (or trends) and temperature values (or trends) before applying further screens.

In a further embodiment, after accepting the first signal for determination of an associated analyte measurement value a correction algorithm is applied to the first signal, for example, by adjusting the first signal using the second signal. In an exemplary adjustment, the correction algorithm comprises correcting the first signal by subtracting at least a portion of the second signal. For example, in some embodiments when the first and second signal are amperometric or coulometric, the correction algorithm comprises Q=Qₐ−kQₚ, where Q is a signal input for determination of an analyte measurement value, Qₐ is the first signal, k is a proportionality factor that is a value between 0 and 1 (and may include the values 0 or 1), and Qₚ is the second signal. As a further example, a correction algorithm comprises correcting the first signal by subtracting at least a portion of the second signal, further taking into account the second signal at a calibration time point. One such exemplary correction algorithm comprises Q=Qₐ−k(Qₚ−Q_{pcal}) where Q is a signal input for determination of an analyte measurement value, Qₐ is the first signal, k is a proportionality factor that is a value between 0 and 1 (and may include the values 0 or 1), Qₚ is the second signal, and Q_{pcal} is the second signal at the calibration time point.

Analytes that can be measured using the microprocessors, methods and devices of the present invention include, but are not limited to, amino acids, enzyme substrates or products indicating a disease state or condition, other markers of disease states or conditions, drugs of abuse (e.g., ethanol, cocaine), therapeutic and/or pharmacological agents (e.g., theophylline, anti-HIV drugs, lithium, anti-epileptic drugs, cyclosporin, chemotherapeutics), electrolytes, physiological analytes of interest (e.g., urate/uric acid, carbonate, calcium, potassium, sodium, chloride, bicarbonate (CO₂), glucose, urea (blood urea nitrogen)., lactate and/or lactic acid, hydroxybutyrate, cholesterol, triglycerides, creatine, creatinine, insulin, hematocrit, and hemoglobin), blood gases (carbon dioxide, oxygen, pH), lipids, heavy metals (e.g., lead, copper), and the like. In a preferred embodiment the analyte is glucose.

The one or more microprocessors of the present invention, in some embodiments, comprises programming to control operating a first sensing device that provides the first signal and operating a second sensing device that provides the second signal. Further, in some embodiments the one or more microprocessors of the present invention comprise programming to control operating a first sampling device (e.g., employing an iontophoretic method) that provides the first sample. The present invention includes analyte monitoring devices that comprise the one or more microprocessors described herein. Such analyte monitoring devices may, for example, comprise one or more microprocessors and first and second electrochemical sensing devices. Further, such analyte monitoring devices may, for example, comprise one or more microprocessors, first and second electrochemical sensing devices, and a sampling device (e.g., an iontophoretic sampling device).

In the context of glucose as an analyte, the ability of the GlucoWatch biographer and other transdermal or transmucosal glucose monitoring systems to detect glucose (as well as transdermal analyte monitoring devices to detect selected analyte) during periods of profuse sweating and/or temperature fluctuations increases the usability and reliability of such devices. For example, when glucose is an analyte of interest, the ability to detect glucose during periods of sweating increases the information available to persons with diabetes on their glycemic condition, and improve the management of diabetes. As the rate of diabetes is increasing in the United States, society will benefit from improved tools for diabetes management as well as decreased health care costs from lower rates of long-term complications enabled by more intensive control of glycemic levels.

2.2.4 Exemplary Embodiments of Passive Collection/Sensing Device Systems of the Present Invention

In a general embodiment, the one or more passive collection/sensing device systems of the present invention comprise a passive collection reservoir capable of being placed in operative contact with a sensing device. Such passive collection reservoir/sensing device systems may be employed in a variety of analyte monitoring devices. In one embodiment, the one or more passive collection reservoirs/sensing devices are typically present in conjunction with one or more active collection reservoirs/sensing devices, wherein the one or more passive collection reservoirs/sensing devices are used to provide information concerning sweat-related analyte and/or temperature changes (e.g., in the subject being monitored). This aspect of the present invention is useful in a variety of analyte monitoring devices that employ minimally invasive or non-invasive sampling methods that rely on methods that increase or enhance transdermal analyte flux, including, but not limited to, iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, and combinations thereof In some embodiments a portion of or the entire surface of a passive collection reservoir may be in contact with a skin surface. Typically the sensing device is in operative contact with the collection reservoir, for example, an electrode assembly comprising a sensing electrode in contact with a hydrogel.

In an alternative embodiment, one or more passive collection reservoirs/sensing devices may be used in combination with a transdermal, spectroscopic method for determination of analyte amount or concentration in a subject being monitored. Accordingly, in one embodiment of this aspect of the present invention, one or more passive collection reservoirs/sensing devices are present in conjunction with a spectroscopic sensing device.

In other embodiments, there may be a layer (e.g., a membrane or mask) between the skin surface and the passive collection reservoir that essentially blocks migration of the analyte into the passive collection reservoir (e.g., where the membrane or mask is substantially impermeable to the analyte). Such embodiments may be employed, for example, to measure signal changes (fluctuations) due to temperature changes (fluctuations). These measured signal changes may also be employed in the compensation and data screening methods of the present invention.

In some embodiments of the present invention, a thermistor may be in operative contact with the passive collection reservoir. Alternatively, a thermistor may be in close proximity to the sensing device that is in operative contact with the passive collection reservoir, or be in-thermal equilibrium with the sensing device, for example, a thermistor may be in close proximity to an electrode assembly comprising a sensing electrode, which is in contact with a hydrogel.

A sampling device and sensing device, comprising one or more passive collection/sensing device systems, may be maintained in operative contact with the skin surface of a biological system to provide frequent measurements. Alternately, the sampling device may be removed and a sensing device, as well as one or more passive collection/sensing device systems, may remain in contact with the biological system to provide frequent measurements.

The present invention also includes methods of manufacturing the passive collection reservoirs/sensing devices of the present invention.

In one aspect, the present invention relates to the use of one or more passive collection reservoir/sensing electrode system in contact with the skin in combination with a previously described one or more active collection reservoir/sensing electrode system for measuring an analyte of interest (see, for example, U.S. Pat. Nos. 6,393,318, 6,341,232, and 6,438,414). This system comprising, for example, a third, passive collection reservoir, is similar to the two-reservoir system in the following respects:

1) A working electrode is present in operative contact with a third collection reservoir to provide an electrochemically reactive surface for peroxide and other electrochemically active compounds.

2) The working electrode area typically covers substantially the same area of skin as is exposed to the collection reservoir.

3) A reference electrode is present to allow proper setting of the working electrode potential relative to the collection reservoir.

4) The collection reservoir is sufficiently ionically conductive to support electrochemical reactions at the working electrode, such as by the addition of sodium chloride in solution.

In one preferred embodiment of the present invention, the collection reservoir electrode system, including a third, passive collection reservoir, comprises:

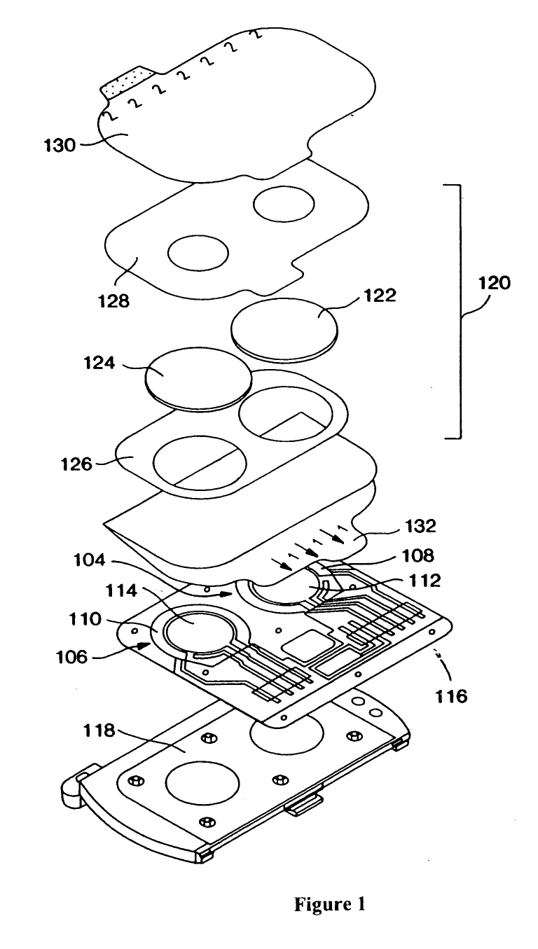

1) A third working electrode fabricated at the same time and from the same materials as the other working electrodes that are being used to determine analyte amount or concentration (for example, the two working electrodes of the AutoSensor shown in the FIG. 1). Thus, the working electrode has substantially the same electrochemical characteristics (e.g., reactivity and temperature response) as the other working electrodes. Also, with this approach the manufacturing cost of adding an additional electrode is reduced, because no additional processing steps is required as compared to fabricating the sensors at different times and with different materials.

2) A collection reservoir associated with the third working electrode is fabricated from the same materials and to the same thickness as other collection reservoirs used by the analyte monitoring device (for example, the two hydrogel collection reservoirs of the AutoSensor shown in the FIG. 1). Thus, the temperature characteristics, diffusion characteristics, and reaction kinetics with analyte (e.g., glucose) is similar to the other collection reservoirs. It is generally desirable that the passive collection reservoir has substantially the same physical characteristics as the active collection reservoir(s). Also, with this approach the manufacturing cost of adding an additional reservoir is reduced, because no additional processing steps is required as compared to fabricating the collection reservoirs at a different times and with different materials. Exemplary materials and methods of making hydrogel collection reservoirs have been previously described (see, e.g., PCT International Publication Nos. WO 97/02811 and WO 00/64533, as well as EP 0 840 597 B1, U.S. Pat. No. 6,615,078, and published U.S. Patent Application No.20040062759).

3) The collection reservoir associated with the third working electrode typically comprises an enzyme, for example, glucose oxidase, that reacts with the analyte (e.g., glucose) to form a chemical signal that can readily react (i.e., is detectable) at the working electrode (e.g., peroxide).

4) The potential of the third working electrode is cycled between a pre-selected potential and open-circuit, in the same way as the other working electrodes, used to provide analyte measurement values, are cycled.

The collection reservoir electrode system comprising a third, passive collection reservoir is different from the two-reservoir system in several important respects, including, but not limited to, the following:

1) Analyte is not actively extracted into the third collection reservoir using a sampling method, for example, no iontophoretic current passes to or from the third collection reservoir electrode system. Accordingly, the third collection reservoir electrode system provides a signal that depends on passive collection of sweat and temperature. Typically the passive collection reservoir/sensing element is not connectable to the iontophoretic circuit.

2) Additional provisions are made for a third applied voltage circuit and current sensing circuit to provide the necessary timed electrical potentials and current measurement functions by the analyte monitoring device.

Although described with reference to a three electrode system, the present invention also includes use of similarly constructed multiple electrode systems, for example, one or more working electrodes associated with collection reservoirs that provide analyte measurement values (i.e., a sample is typically extracted into the collection reservoir), and one or more working electrodes associated with collection reservoirs that rely on passive extraction of the analyte of interest.

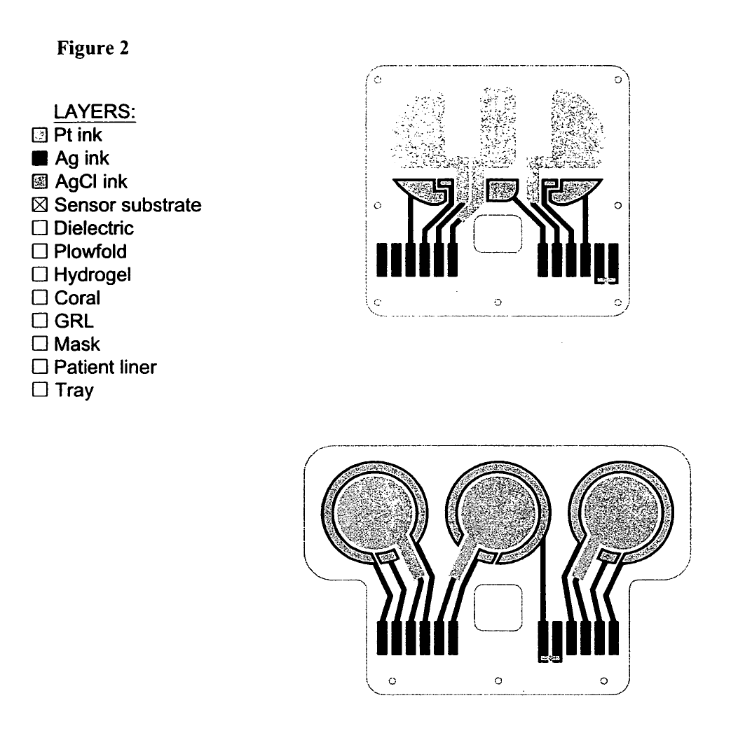

FIGS. 2-11 schematically present preferred embodiments of the present invention. The embodiments shown are exemplary collection assemblies/electrode assemblies comparable to the AutoSensor shown in FIG. 1; however, each of the embodiments of FIGS. 2-11 comprise a third, passive collection reservoir in addition to the two active reservoirs shown in FIG. 1. Each figure presents two preferred embodiments, wherein a first embodiment is presented in the top portion of each figure and a second embodiment is presented in the lower portion of the figure. Each figure in series (i.e., from FIG. 2 through FIG. 11) highlights different layers of the total assemblies, thus providing guidance for to how assemble the devices. The layers shown in each drawing are indicated by an “X”, or a filled box in the legend titled “Layers,” wherein an X indicates the outline geometry of the component is shown. A filled box indicates the component is hatched, thus clarifying where material is present and not present for that component. Because all components (except for the tray) are thin compared to their extent, only plan views of the assemblies are shown. The figures are presented and discussed in the order in which they can be assembled. These figures are for purposes of illustration only, other embodiments of the present invention will be apparent to one of ordinary skill in the art in view of the teachings of the present specification.

In FIGS. 2-11, there are two alternative designs shown for the collection assemblies/electrode assemblies, one on the top portion of each page and one on the bottom portion of the page. The design on the bottom is typically used for laboratory, modeling, and experimental work. It minimizes the differences between the passive electrode and the active electrodes, that is, the geometry is the same for electrodes, gels, area of skin exposed to gel, as well as the amount of silver is the same. The collection assembly/electrode assembly design on the top of each page is more compact, thus providing a lower cost of materials for manufacturing. The small horizontal bar (e.g., in the embodiment at the top of the figures the small grey horizontal bar connecting two dark black vertical bars all the way to the bottom/right at the bottom of the drawing, and in the embodiment at the bottom of the figures the small grey horizontal bar connecting two dark black vertical bars bottom/right of center) provides compatibility of these embodiments of the collection assembly/electrode assembly with the current electronics of the GlucoWatch biographer and the GlucoWatch biographer G2. These devices perform a continuity check to verify the presence of a collection assembly/electrode assembly (e.g., an AutoSensor) snapped into place on the back of the devices.

FIG. 2 illustrates the screen printed sensor inks on a sensor substrate. The Pt ink electrodes are the working electrodes for each collection reservoir. The large electrodes with silver and silver-chloride inks are the counter electrodes. The small electrodes with silver and silver-chloride inks are the reference electrodes. The sensors are shown in this drawing in a flat as-printed configuration.

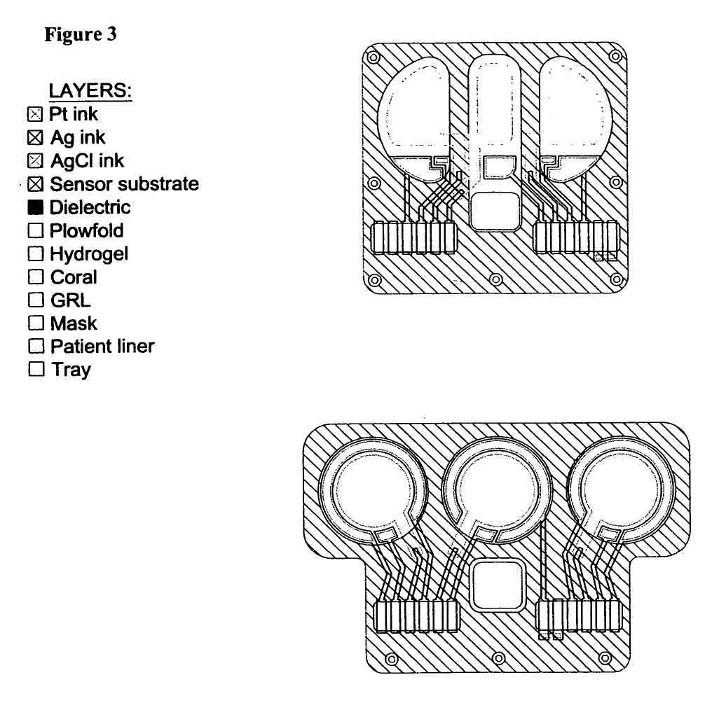

FIG. 3 illustrates a dielectric layer, added on top of the printed sensor, that provides electrical insulation in the areas covered by the dielectric layer. Again, the sensors are shown in the flat configuration.

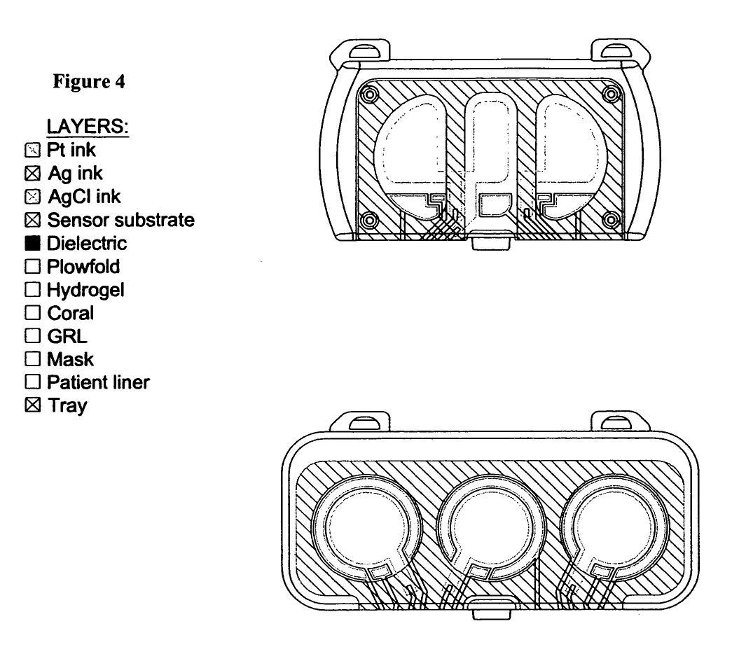

FIG. 4 illustrates the sensors after wrapping around the tray and staking or otherwise adhering the sensor to the tray. The side of the collection assembly/electrode assembly that contacts the skin is shown.

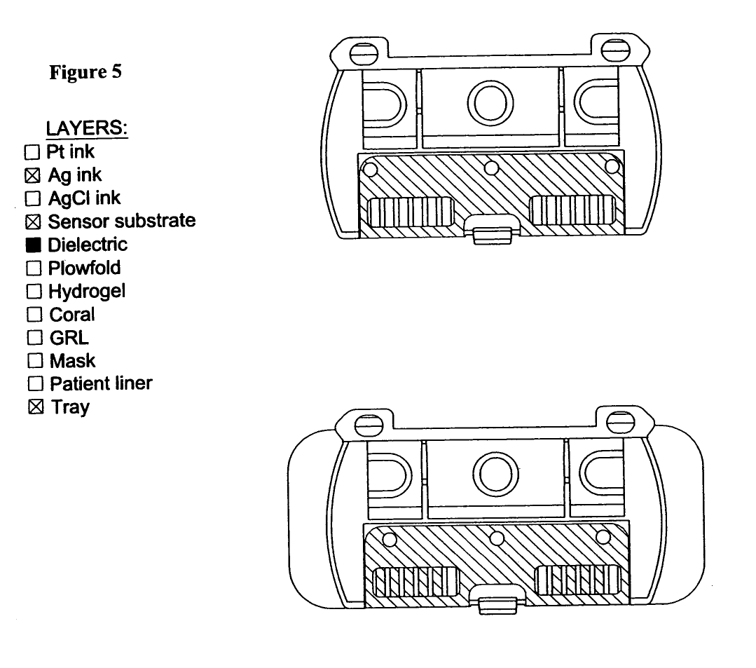

FIG. 5 is the same as FIG. 4, except the side facing away from the skin is shown.

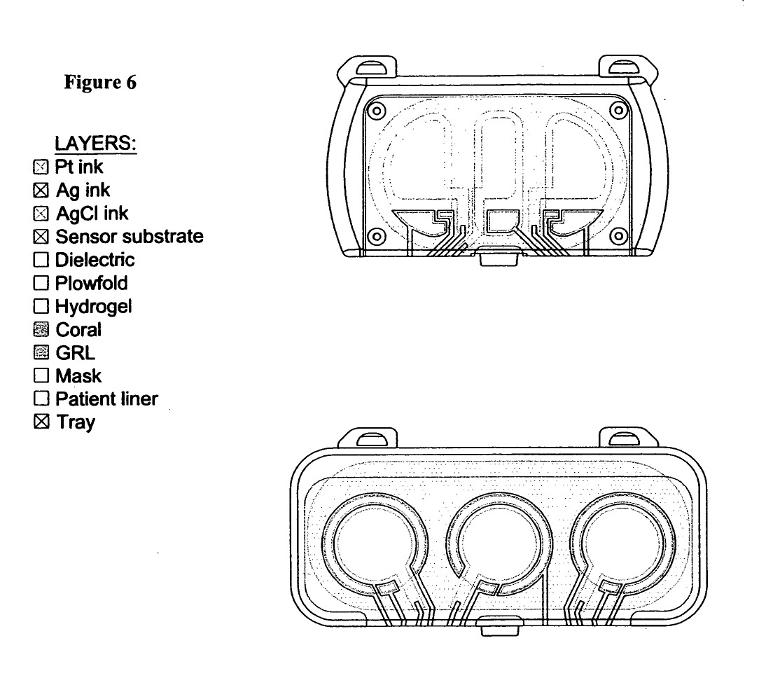

FIG. 6 illustrates the gel retaining layer (GRL) or corral attached to the sensor. This layer has adhesive on two sides, thus attaching to the sensor, and to the mask, once it is placed in position. The electrodes and the hydrogels are typically aligned with openings defined by the GRL. If a corral is used, it typically provides containment means to hold the ionically conductive material.

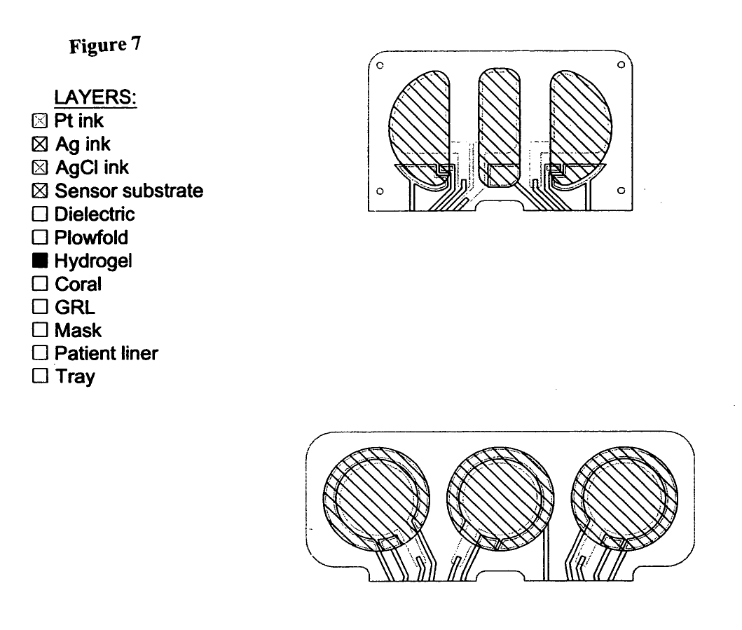

FIG. 7 illustrates the hydrogel discs (in this embodiment the hydrogel disks are the collection reservoirs) placed in position. The hydrogels cover the necessary areas on the sensor (typically the Ag/AgCl and Pt electrodes do not contact the skin or mucosal surface; the hydrogel provides the contact between the skin or mucosal surface and these electrodes).

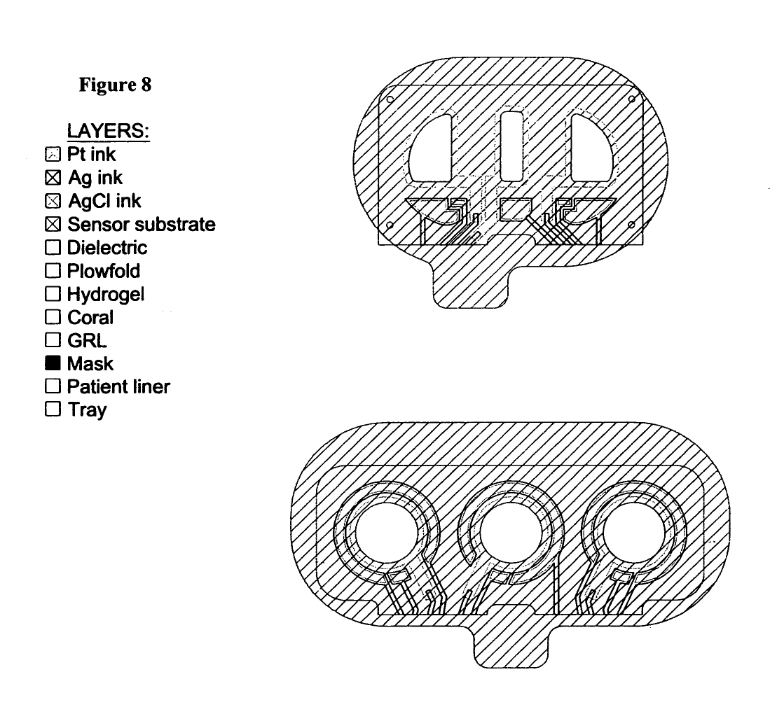

FIG. 8 illustrates the mask layer placed in position over the sensor. Openings defined by the mask layer leave portions of the hydrogel exposed to the skin or mucosal surface, once placed on the subject. The skin side of the mask is coated with adhesive to provide means of attachment to the skin. The electrodes and the hydrogels are typically aligned with openings defined by the mask layer.

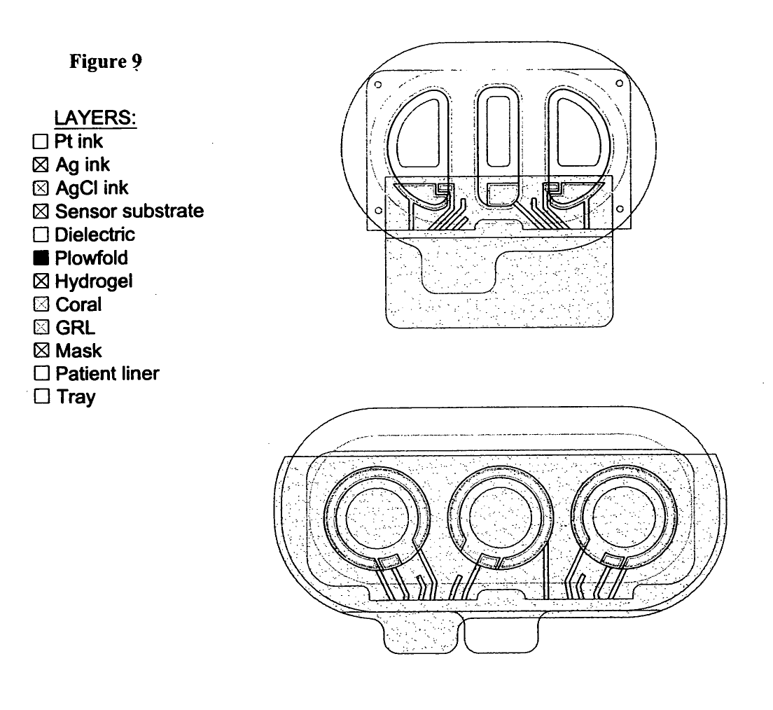

FIG. 9 illustrates a removable plowfold liner layer that separates the hydrogels from the electrode assembly (e.g., the Pt, and silver and silver-chloride electrodes) during storage.

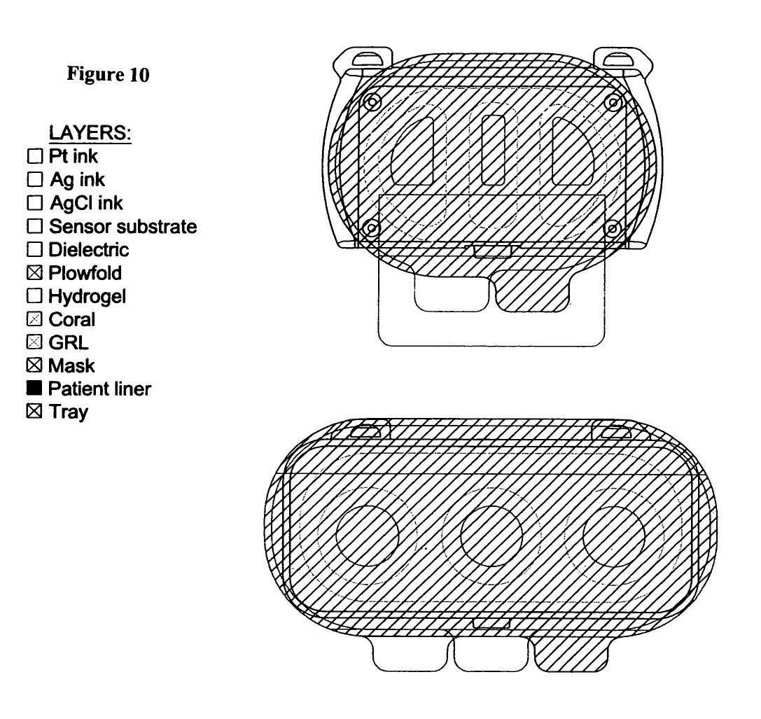

FIG. 10 illustrates a removable patient liner that covers the hydrogels and the adhesive on the mask. This liner typically prevents dry-out of the hydrogels during handling.

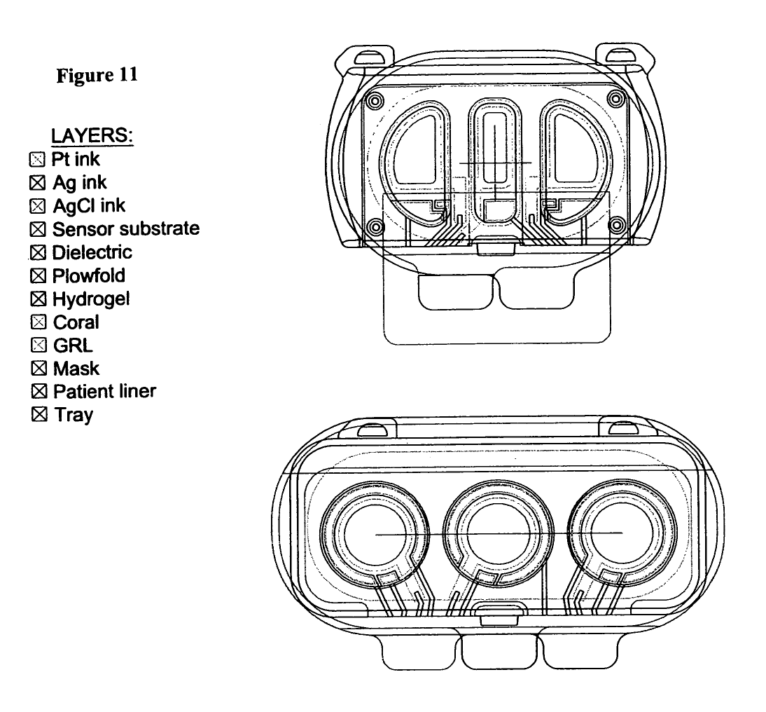

FIG. 11 illustrates all the layers simultaneously that comprise the total collection assembly/electrode assembly for the two preferred embodiments that are shown in this series of figures.

Not shown in the figures are the electronics portions of (i) the sensing system that applies and reads electrical signals to and from the collection assembly/electrode assembly, (ii) the sampling system that provides current for iontophoretic extraction, (iii) the analyte monitoring device that allows inputs from the user, displays results to the user, and sends automatic alerts. In these preferred embodiments shown, the collection assembly/electrode assembly and analyte monitoring device electronics are designed such that the two can snap together prior to use.

In one aspect, the present invention relates to collection assemblies/electrode assemblies for use in an analyte monitoring device. More particularly, in one embodiment the present collection assemblies/electrode assemblies are used in analyte monitoring devices employing transdermal or transmucosal sampling methods, for example, wherein the sampling device is placed in operative contact with a skin or mucosal surface of a biological system to obtain a chemical signal associated with one or more analytes of interest. One exemplary sampling device transdermally or transmucosally extracts the analyte from the biological system using an iontophoretic sampling technique. Other sampling techniques may be employed as well including, but not limited to, iontophoresis (including reverse iontophoresis and electroosmosis), sonophoresis, microdialysis, suction, electroporation, thermal poration, use of microporation (e.g., by laser or thermal ablation), use of microneedles, use of microfine lances, microfine cannulas, skin permeabilization, chemical permeation enhancers, use of laser devices, and combinations thereof. A sampling device and sensing device may be maintained in operative contact with the skin or mucosal surface of the biological system to provide frequent measurements. Alternately, the sampling device may be removed and a sensing device may remain in contact with the biological system to provide frequent measurements, for example, continual or continuous analyte measurement.

One embodiment of the invention provides a collection assembly/electrode assembly comprising a tri-layer collection assembly for use in an analyte monitoring device. The collection assembly is formed from a series of functional layers including: (1) a first surface layer, for example, a mask layer, that is comprised of a substantially planar material that defines three or more openings that extend there through; (2) a second surface layer, for example, a gel retaining layer, that is also comprised of a substantially planar material and defines three or more openings therein; and (3) an intervening layer that is positioned between the first and second surface layers, wherein the intervening layer is comprised of an ionically conductive material having three or more portions. The first and second surface layers overlap the intervening layer at corresponding positions, and contact each other at their corresponding overlaps, such overlaps can be used to form a laminate structure. The openings in the first and second surface layers are axially aligned to provide a flow path through the laminate (i.e., a flow path that extends between the two surfaces and passes through the intervening layer). The overhangs provided by the mask and gel retaining layers are generally contacted with each other to sandwich the collection insert there between and form a collection assembly. The collection assembly is placed in operative combination with an electrode assembly by aligning the openings of the gel retaining layer with the electrodes of the electrode assembly to form an a collection assembly/electrode assembly. The collection assembly/electrode assembly can further be placed in a support tray.

In one embodiment, the invention is directed to a collection assembly/electrode assembly for use in an analyte monitoring device comprising an iontophoretic sampling device useful to monitor an analyte present in a biological system. The collection assembly/electrode assembly comprises:

(I) a collection assembly which comprises,

(a) a collection insert layer comprised of an ionically conductive material having first, second, and third portions, for example, three hydrogels, each portion having first and second surfaces,

(b) a mask layer comprised of a substantially planar material that is substantially impermeable to the one or more selected analytes or derivatives thereof, wherein the mask layer (i) has inner and outer faces and the outer face provides contact with the biological system and the inner face is positioned in facing relation with the first surface of the collection insert layer, (ii) defines first, second, and third openings that are aligned with the first, second, and third portions of the collection insert layer, (iii) each opening exposes at least a portion of the first surface of one of the portions of the collection insert layer, and (iv) has a border which extends beyond the first surface of each portion of the collection insert layer to provide an overhang;

(c) a gel retaining layer having (i) inner and outer faces wherein the inner face is positioned in facing relation with the second surface of the collection insert layer, (ii) defines first, second, and third openings that are aligned with the first, second, and third portions of the collection insert layer, (iii) each opening exposes at least a portion of the second surface of one of the portions of the collection insert layer, and (iv) has a border which extends beyond the first surface of each portion of the collection insert layer to provide an overhang; and

(d) wherein the first, second, and third openings in the mask layer are positioned in the collection assembly such that they are aligned with the first, second, and third openings in the gel retaining layer and thereby define a plurality of flow paths through said collection assembly;

(II) an electrode assembly having an inner and outer face, the inner face comprising first, second, and third electrodes, wherein the first, second, and third electrodes are aligned with the first, second, and third openings in the gel retaining layer of the collection assembly; and

(III) a support tray that contacts the outer face of the electrode assembly.

In an alternative embodiment, the invention is directed to an a collection assembly/electrode assembly comprising:

(I) a collection assembly which comprises,

a) a collection insert layer comprised of an ionically conductive material having first, second, and third portions, for example, three hydrogels, each portion having first and second surfaces,

b) a mask layer comprised of a substantially planar material that is substantially impermeable to the one or more selected analytes or derivatives thereof, wherein the mask layer (i) has inner and outer faces and the outer face provides contact with the biological system and the inner face is positioned in facing relation with the first surface of the collection insert layer, (ii) defines first and second openings that are aligned with the first and second portions of the collection insert layer, wherein each opening exposes at least a portion of the first surface of one of the portions of the collection insert layer, (iii) the inner face of the mask contacts the first surface of the third portion of the collection layer, and (iv) has a border which extends beyond the first surface of each portion of the collection insert layer to provide an overhang;

(c) a gel retaining layer having (i) inner and outer faces wherein the inner face is positioned in facing relation with the second surface of the collection insert layer, (ii) defines first, second and third openings that are aligned with the first, second, and third portions of the collection insert layer, (iii) each opening exposes at least a portion of the second surface of one of the portions of the collection insert layer, and (iv) has a border which extends beyond the first surface of each portion of the collection insert layer to provide an overhang; and

(d) wherein the first and second openings in the mask layer are positioned in the collection assembly such that they are aligned with the first and second openings in the gel retaining layer and thereby define a plurality of flow paths through said collection assembly;

(II) an electrode assembly having an inner and outer face, the inner face comprising first, second, and third electrodes, wherein the first, second, and third electrodes are aligned with the first, second, and third openings in the gel retaining layer of the collection assembly; and

(III) a support tray that contacts the outer face of the electrode assembly.

In some embodiments of the collection assembly/electrode assembly (e.g., AutoSensor assembly) of the present invention, the first, second, and third electrodes are all bimodal electrodes, wherein two of the electrodes are used to pass iontophoretic current and the third electrode does not pass iontophoretic current, that is, the first and second bimodal electrodes are connectable to an iontophoretic circuit and the third electrode is not connectable to the iontophoretic circuit but the third electrode can perform as a sensing electrode.

In further embodiments, the collection assembly/electrode assembly (e.g., AutoSensor assembly) comprise a first removable liner attached to the outer face of the gel retaining layer, and/or a second removable liner attached to the outer face of the mask layer. In addition, a plowfold liner can be used, for example, between the electrode surfaces and the collection inserts.

In further embodiments, the invention is directed to a sealed package containing the collection assembly/electrode assembly (e.g., AutoSensor assembly) described above. The sealed package may also contain a hydrating insert.

The present invention also includes methods of manufacturing the collection assemblies/electrode assemblies of the present invention.

Electrode assemblies of the present invention can be formulated using methods known in the art in view of the teachings of the present invention. For example, the electrode assemblies of the present invention may be printed providing a substantially uniform deposition of a conductive polymer composite film (e.g., an electrode ink formulation) onto one surface of a substrate (i.e., the base support). It will be appreciated by those skilled in the art that a variety of techniques may be used to effect substantially uniform deposition of a material onto a substrate, e.g., Gravure-type printing, extrusion coating, screen coating, spraying, painting, electroplating, laminating, or the like. See, for example, “Polymer Thick Film, by Ken Gilleo, New York:Van Nostrand Reinhold, 1996, pages 171-185.

Once formulated, the Ag/AgCl electrode composition and a sensing electrode composition are typically affixed to a suitable rigid or flexible nonconductive surface (for example, polyester, polycarbonate, vinyl, acrylic, PETG (polyethylene terephthalate copolymer), PEN, and polyimide). In one embodiment of the present invention, the electrode assemblies can include bimodal electrodes. Exemplary suitable sensing electrode materials, sensing electrodes, and methods of making same have been previously described (see, e.g., EP 0 942 278, GB 2 335 278, U.S. Pat. No. 6,587,705, Published U.S. Patent Application No. 20030155557, and PCT International Publication No. WO 03/054070).

The collection assembly/electrode assembly of the present invention typically comprises a collection assembly that may include, for example:

a) a mask layer comprised of a substantially planar material that is substantially impermeable to the selected analyte or derivatives thereof, where the mask layer defines a plurality of openings, has inner and outer faces, and the outer face provides contact with the biological system;

b) a collection insert layer comprised of a plurality of portions of an ionically conductive material having first and second surfaces, and

c) a gel retaining layer comprised of a substantially planar material that is substantially impermeable to the selected analyte or derivatives thereof, where the gel retaining layer defines a plurality of openings, has inner and outer faces, and the outer face contacts the electrode assembly, wherein the mask layer, gel retaining layer, and collection insert layer are configured such that (i) at least a portion of the collection insert is exposed to provide contact with the biological system, and (ii) flow of the analyte through the first surface of the collection insert layer from the biological system is prevented by the mask layer for any portion of the first surface of the collection insert layer that is in contact with the inner face of the mask layer. Such collection assemblies can be included in a collection assembly/electrode assembly that typically comprises (a) the collection assembly, (b) an electrode assembly having an inner face comprising an electrode and an outer face, where the inner face of the electrode assembly and the collection assembly are aligned to define a plurality of flow paths through said collection assembly, and (c) a support tray that contacts the outer face of the electrode assembly.

In one embodiment, the mask layer and gel retaining layer each define three or more openings and at least a part of a portion of a collection insert layer is exposed by each opening to provide a flow path through the collection assembly. Neither the mask layer nor the gel retaining layer are required by the present invention. Any containment means for the collection insert may be used. For example, the collection insert may be contained by a corral or gasket that contains, seals, or retains the collection insert at a desired location. The entire surface of the collection insert may be exposed to the skin surface, for example, when a gasket or corral is used. Mask and gel retaining layers may be used with a gasket or corral and in this case the mask and gel retaining layers typically contacts the edges of the gasket or corral.

In another embodiment, the mask layer may cover the third portion of the collection insert layer. In this embodiment, transport of the analyte to the sensing electrode is blocked and a sensing electrode in contact with the third portion provides information about biosensor signal changes due to temperature changes/fluctuations. A thermistor may also be in contact with such a third portion.

The mask layer may be coated with an adhesive on either of its faces or on both of its faces. Further, a liner maybe adhered to one of the faces of the mask layer, typically the outer face—similarly for the gel retaining layer. In one embodiment, (i) the outer face of the mask layer has an adhesive coating and a liner attached, (ii) the inner face of the mask layer contacts the collection inserts and adheres to the inner face of the gel retaining layer, and (iii) the outer face of the gel retaining layer is adhered to a second liner (e.g., a plow-fold liner).

The collection assemblies may be prepared as laminates. Further, other components, such as support trays and electrodes or electrode assemblies can be combined with the collection assemblies or laminates to form, for example, AutoSensor assemblies.

Further, the collection assemblies/electrode assemblies of the invention may be provided in sealed packages. In some embodiments, such sealed packets further comprise a source of hydration (e.g., a hydrating insert) which ensures that the collection inserts will not dehydrate prior to use.

The collection assemblies/electrode assemblies (e.g., AutoSensors) of the present invention are particularly well suited for use as consumable components in an analyte monitoring device comprising an iontophoretic sampling device. In one embodiment a collection assembly is aligned with an electrode assembly that includes both iontophoretic and sensing electrodes. A tray is adapted to hold the electrodes and collection assemblies in operative alignment, and provides electrical connection between the electrode assembly and control components provided by an associated housing element. If desired, the tray can be comprised of a substantially rigid substrate and have features or structures which cooperate and/or help align the various assemblies in the sampling device. For example, the tray can have one or more wells or recesses, and/or one or more lips, rims, or other structures which depend from the substrate, each of which features or structures facilitate register between the electrode assembly, the collection assembly and the associated components of the sampling device. The tray can be composed of any suitable material, desirable characteristics of which can include the following: (i) high heat distortion temperature (to allow hot melt bonding of the electrode assembly to the tray, if necessary or desired); (ii) optimum rigidity, to allow for ease of handling and insertion into the housing of the monitoring device; (iii) low moisture uptake, to insure that proper hydration of the ionically conductive medium (e.g., hydrogel collection inserts) is maintained when the medium is stored in proximity to the tray; and, (iv) moldable by conventional processing techniques, for example, injection molding.

Materials for use in manufacturing the tray include, but are not limited to, the following: PETG (polyethylene terephthalate copolymer); ABS (acrylonitrile-butadiene-styrene co-polymer); SAN (styrene-acrylonitrile copolymer); SMA (styrene-maleic anhydride copolymer); HIPS (high impact polystyrene); polyethylene terephthalate (PET); polystyrene (PS); polypropylene (PP); and blends thereof. In a preferred embodiment the tray is formed from high impact polystyrene.

The electrode assembly is typically fixed to the tray to, for example, facilitate register between the electrode assembly and the associated components of the housing of the analyte monitoring device. The electrode assembly may be manufactured as part of the tray, or, the electrode assembly may be attached to the tray by, for example, (i) using connecting means that allow the electrode assembly to engage the tray (e.g., holes in the electrode assembly with corresponding pegs on the tray); or (ii) use of an adhesive. Exemplary adhesives include, but are not limited to, the following: acrylate, cyanoacrylate, styrene-butadiene, co-polymer based adhesives, and silicone. In a preferred embodiment the tray is attached to the electrode assembly as in (i) above with the pegs deformed, thus locking the components together.

The collection assembly typically includes three or more collection inserts that are comprised of an ionically conductive material (e.g., hydrogels). Each collection insert has first and second opposing surfaces. The collection insert is preferably comprised of a substantially planar hydrogel disk. The first opposing surface of the insert is intended for contact with a target surface (skin or mucosa), and the second opposing surface is intended for contact with the electrode assembly, thereby establishing a flow path between the target surface and the selected electrodes. A mask layer is positioned over the first surface of the collection insert. The mask layer comprises three or more openings that are sized to expose at least a portion of the first surface of the corresponding, aligned hydrogel of the collection insert layer. A border region of the mask layer generally extends beyond the first surface of the collection insert to provide an overhang.

A gel retaining layer is positioned in facing relation with the second surface of the collection insert layer. The gel retaining layer has three or more openings that expose at least a portion of the second surface of the corresponding, aligned hydrogel of the collection insert layer. A border region of the gel retaining layer extends beyond the second surface in order to provide an overhang. The overhangs provided by the mask and gel retaining layers serve as a point of attachment between the two layers. When these layers are attached to each other at their overhanging portions, a laminate is formed wherein the collection insert is sandwiched between the two layers to provide a three-layer structure. Although the overhangs provided by border regions may extend along an edge of the mask and gel retaining layers, the overhangs can, of course, be formed from one or more corresponding tab overhangs (positioned anywhere on the subject layers), one or more corresponding edges (opposite and/or adjacent edges), or can be formed from a continuous overhang which encompasses the collection insert (e.g., an overhang which circumscribes an oval- or circular-shaped insert, or an overhang which surrounds all sides of a square-, rectangular-, rhomboid-, or triangular-shaped insert).

The three or more openings in the mask layer, and the three or more openings in the gel retaining layer can have any suitable geometry that is generally dictated by the shape of the collection insert and/or the shape of the electrodes used in the electrode assembly. In the embodiment depicted in the lower portion of FIG. 11, wherein the electrodes are arranged in a circular configuration and the collection insert is a circular disk, the openings preferably have a round, oval, ellipsoid, or “D”-shape which serves to collimate the flow (i.e., reduce or eliminate the edge effect flow) of chemical signal as it passes through the collection assembly toward the electrode assembly.

The openings in the mask and gel retaining layers can be sized the same or differently, wherein the particular sizes of the openings may generally be set by the overall surface area of the sensing electrode that the collection assembly must operate with in the sensing device. Although the collection assemblies of the present invention can be provided in any size suitable for a targeted skin or mucosal surface, an assembly that is used with an analyte monitoring device that contacts a subject's wrist will generally have a surface area on each face in the range of about 0.5 cm² to 15 cm². The openings generally expose about 50% of the area of the sensing electrode, within a manufacturing tolerance of about ±20%. In general, the openings constitute an area that is in the range of 1% to 90% of the surface area encompassed by the mask or gel retaining layer plus the opening(s). The openings are, however, sized smaller than the overall surface of the collection insert in at least one dimension.

The size or geometric surface area of the sensing electrode, the thickness of the collection insert, the sizes of the openings in the mask and gel retaining layers, and the size of the overhangs provided by border regions of the mask and gel retaining layers are all interrelated to each other. For example, when the thickness of the collection insert is increased, the size of the opening may be decreased to obtain the same degree of reduction of edge effect flow (radial transport) of a transported analyte. However, it is typically desirable to maximize the size of the openings in order to maximize the amount of analyte (or related chemical signal) that contacts the reactive surface of the sensing electrode.

The physical characteristics of the mask and gel retaining layers are selected so as to optimize the operational performance of the collection assembly. More particularly, because the assembly is intended to be contacted with a target surface for an extended period of time, the layers preferably have sufficient mechanical integrity so as to provide for such extended use. Furthermore, the layers should have sufficient flex and stretch-ability so as to resist tearing or rupture due to ordinary motion in the target surface, for example, movement of a subjects arm when the analyte monitoring device is contacted with a forearm or wrist. The layers can also have, for example, rounded comers that tolerate a greater degree of twist and flex in a target area (without breaking contact) than layers which have sharp, angular comers. The layers also provide for some degree of sealing between the target surface and the collection assembly, as well as between the collection assembly and the electrode assembly, and can provide for electrical, chemical, and/or electrochemical isolation between multiple collection inserts in the collection assembly and their corresponding electrodes in the electrode assembly. Other physical characteristics include the degree of occlusivity provided by the mask layer, adhesion to the target surface and/or electrode assembly, and mechanical containment of the associated collection insert(s). In one embodiment, the collection assembly includes three hydrogels (as depicted in FIG. 11), and the mask and gel retaining layers have corresponding central regions that are disposed between corresponding openings in the layers and provide for a further point of attachment between the two layers. As will be appreciated by the skilled artisan upon reading the present specification, this further point of attachment provides for chemical and electrical isolation between the two collection inserts.

The mask and gel retaining layers are preferably composed of materials that are substantially impermeable to the analyte (also, typically, to chemical signal) to be detected (e.g., glucose); however, the material can be permeable to other substances. By “substantially impermeable” is meant that the material reduces or eliminates analyte and corresponding chemical signal transport (e.g., by diffusion). The material can allow for a low levels of analyte and/or chemical signal transport, with the proviso that analyte and/or chemical signal that passes through the material does not cause significant edge effects at the sensing electrode used in conjunction with the mask and gel retaining layers. Examples of materials that can be used to form the layers include, without limitation, the following: polymeric materials—such as, polyethylene (PE) {including, high density polyethylene (HDPE), low density polyethylene (LDPE), and very low density polyethylene (VLDPE)}, polyethylene copolymers, thermoplastic elastomers, silicon elastomers, polyurethane (PU), polypropylene (PP), (PET), nylon, flexible polyvinylchloride (PVC), and the like; natural rubber or synthetic rubber, such as latex; and combinations of the foregoing materials. Of these materials, exemplary flexible materials include, but are not limited to, the following: HDPE, LDPE, nylon, PET, PP, and flexible PVC. Stretchable materials include, but are not limited to, VLDPE, PU, silicone elastomers, and rubbers (e.g., natural rubbers, synthetic rubbers, and latex). In addition, adhesive materials, for example, acrylate, styrene butadiene rubber (SBR) based adhesives, styrene-ethylene-butylene rubber (SER) based adhesives, and similar pressure sensitive adhesives, can be used to form layers as well.

Each layer can be composed of a single material, or can be composed of two or more materials (e.g., multiple layers of the same or different materials) to form a chemical signal-impermeable composition.

Methods for making the mask and gel retaining layers include, without limitation, extrusion processes, flow and form molding techniques, die cutting, and stamping techniques, which are all practiced according to methods well known in the art. Most preferably, the layers are manufactured in a manner that is the most economical without compromising performance (e.g., impermeability to a chemical signal, the ability to manipulate the layers by hand without breaking or otherwise compromising operability, and the like). The layers may further have an adhesive coating (e.g., a pressure sensitive adhesive) on one or both surfaces. Exemplary adhesives include, but are not limited to, the following: starch, acrylate, styrene butadiene rubber-based, silicone, and the like. Adhesives that may come in contact with skin have a toxological profile compatible with skin-contact. In an exemplary embodiment, SBR-adhesive RP100 (John Deal Corporation, Mount Juliet, Tenn.) can be used on both sides of a 0.001 inch thick PET film (Melinex #329, DuPont) gel retaining layer to adhere to the mask and the other side to the sensor. Another exemplary embodiment uses acrylate #87-2196 (National Starch and Chemical Corporation, Bridgewater, N.J.) on the skin side of a 0.002 inch thick polyurethane (e.g., Dow Pellethane; Dow Chemical Corp., Midland, Mich.) mask to adhere the mask to the skin. Further, the mask and gel retaining layers may be coated with a material which absorbs one or more compounds or ions that may be extracted into the collection insert during sampling.

Because the collection assemblies/electrode assemblies (e.g., AutoSensor assemblies) of the present invention are intended for use as consumable (replaceable) components for an analyte monitoring device, the various constituents of the assemblies are preferably manufactured and then pre-assembled in an easy-to-use structure that can be inserted and then removed from the analyte monitoring device housing by the consumer. In this regard, after the mask layer, gel retaining layer, and collection insert(s) are produced, they are aligned as shown in FIG. 11, and the overhangs provided by borders of the mask and gel retaining layers are attached to each other to provide a three-layer laminate that sandwiches the collection insert in between the mask and gel retaining layers as described above. The resulting collection assembly is then placed in operative alignment with the electrodes of an electrode assembly to form the collection assembly/electrode assembly (e.g., AutoSensor assembly), which may further be placed in a support tray.

If desired, packages comprising the collection assembly/electrode assembly can include a source of hydration (e.g., a hydrating insert formed from a water-soaked pad, non-woven material, or gel that ensures that the collection inserts will not dehydrate prior to use. The hydrating insert may include other components as well, such as, buffers and antimicrobial compounds. The source of hydration is disposed of after the collection assembly/electrode assembly has been removed from the package, and thus does not typically form a component of the analyte monitoring device.

The pre-assembled collection assemblies/electrode assemblies (e.g., AutoSensor assemblies) can include one or more optional liners which facilitate handling of the assembly. For example, a removable patient liner can be applied over the mask layer, particularly when the mask layer is coated with an adhesive. An additional removable liner can be applied over the gel retaining layer (e.g., a plow-fold liner). The removable liners are intended to remain in place until just prior to use of the assembly, and are thus manufactured from any suitable material which will not be too difficult to remove, but which will remain in place during packaging, shipment and storage to provide added protection to the assembly. If the mask and/or gel retaining layers are coated with (or actually formed from) an adhesive, the removable liners can preferably be comprised of a polypropylene or treated polyester material which does not adhere well to commonly used contact adhesives. Other suitable materials include, without limitation, water and/or solvent impermeable polymers (including, but not limited to PET, PP, PE, and the like) and treated metal foils.

The removable liners are generally shaped to cover the outer surfaces of the mask and gel retaining layers. The liners can further include grasping means, such as a tab, and intuitive indicia (such as numbering) that indicates the order in which the liners are intended to be removed prior to use in the analyte monitoring device. If desired, the liners can be shaped in a folded “V” (e.g., a “plow-fold” liner) or “Z” shape that provides a grasping means for the user, as well as providing for a controlled release motion in the liner. Alternatively, the liners can have an internal cut (e.g., a spiral cut extending from one edge of the liner and ending in the surface of the liner) or a scoring pattern which facilitates removal of the liner. Particularly, the liner material, shape, and related cuts or patterns or weakness are selected to ensure that removal of the liners does not disrupt the alignment between the various components of the collection assembly/electrode assembly.

In one aspect, as described herein, the present invention relates to an analyte monitoring device comprising, (A) one or more collection reservoirs adapted for contact with a skin or mucosal surface of a subject, wherein (i) movement of the analyte into the collection reservoirs is enhanced by a transdermal or transmucosal sampling method, and (ii) during use of the device at least one collection device is placed in operative contact with an analyte sensing device; and (B) one or more collection reservoirs adapted for contact with a skin or mucosal surface of a subject, wherein (i) movement of the analyte into the collection reservoirs not enhanced by the transdermal or transmucosal sampling method, and (ii) during use of the device at least one collection device is placed in operative contact with an analyte sensing device. In one embodiment, during use of the device at least one collection reservoir of (B) is in contact with a thermistor.

In a preferred embodiment, the physical characteristics of at least one collection reservoir of (A) are substantially the same as the physical characteristics of at least one collection reservoir of (B). An exemplary collection reservoir is a hydrogel.

In some embodiments, the analyte monitoring device comprises an analyte sensing device that detects analyte electrochemically. Such a device typically comprises a sensing electrode. In a preferred embodiment, the physical characteristics of the sensing electrode in contact with at least one collection reservoir of (A) has substantially the same physical characteristics of the sensing electrode in contact with at least one collection reservoir of (B). Further, in some embodiments the analyte sensing device comprises an enzyme to facilitate electrochemical detection of the analyte (e.g., when the analyte is glucose and the enzyme comprises glucose oxidase).

In one embodiment the analyte monitoring device further comprises iontophoretic electrodes in contact with the one or more collection reservoirs of (A). The device may also comprise iontophoretic electrodes in contact with the one or more collection reservoirs of (B) but in this case the iontophoretic electrodes are typically not connectable to the iontophoretic circuit, that is the iontophoretic electrodes are not activatable to use for extraction.

In yet another embodiment, a collection reservoir of (B) of the analyte monitoring device comprises first and second surfaces, the first surface is in contact with a sensing device and the second surface is in contact with a membrane substantially impermeable to analyte, and the membrane is adapted for contact with the skin or mucosal surface.

The present invention also includes methods of manufacturing the collection assemblies, collection/electrode assemblies, AutoSensors, and devices of the present invention.

3.0.0 Trainable Algorithms Employing the Methods of the Present Invention

In one aspect of the present invention, trainable algorithms may be applied to the methods of the present invention, for example, methods for improved selectivity of data screens and/or methods of compensation for the effects of sweat and/or temperature change. Mathematical, statistical and/or pattern recognition techniques can be applied to the methods of the present invention, including, but not limited to, neural networks, genetic algorithm signal processing, linear regression, multiple-linear regression, non-linear regression methods, estimation methods, or principal components analysis of statistical (test) measurements. Training data (e.g., data sets obtained from measurements of an analyte monitoring device) may be used to determine the unknown parameters. In one particular embodiment, the methods can be carried out using artificial neural networks or genetic algorithms. The structure of a particular neural network algorithm used in the practice of the invention can vary widely; however, the network should contain an input layer, one or more hidden layers, and one output layer. Such networks can be trained on a test data set, and then applied to a population. In another embodiment, training data is used to determine unknown parameters in the Mixtures of Experts (MOE) algorithm using the Expectation Maximization Method. The Mixtures of Experts algorithm is typically trained until convergence of the weights was achieved. There are an numerous suitable network types, transfer functions, training criteria, testing and application methods which will occur to the ordinarily skilled artisan upon reading the instant specification. In another embodiment, a decision tree (also called classification tree) may be employed that utilizes a hierarchical evaluation of thresholds (see, for example, J. J. Oliver, et. al, in Proceedings of the 5th Australian Joint Conference on Artificial Intelligence, pages 361-367, A. Adams and L. Sterling, editors, World Scientific, Singapore, 1992; D. J. Hand, et al., Pattern Recognition, 31(5):641-650, 1998; J. J. Oliver and D. J. Hand, Journal of Classification, 13:281-297, 1996; W. Buntine, Statistics and Computing, 2:63-73, 1992; L. Breiman, et al., “Classification and Regression Trees” Wadsworth, Belmont, Calif., 1984; C4.5: Programs for Machine Learning, J. Ross Quinlan, The Morgan Kaufmann Series in Machine Learning, Pat Langley, Series Editor, October 1992, ISBN 1-55860-238-0). Commercial software for structuring and execution of decision trees is available (e.g., CART (5), Salford Systems, San Diego, Calif.; C4.5 (6), RuleQuest Research Pty Ltd., St Ives NSW Australia; and Dgraph (1,3), Jon Oliver, Cygnus, Redwood City, Calif.) and may be used in the methods of the present invention in view of the teachings of the present specification.

Some simple versions of decision trees based on the methods of the present invention are as follows. First, threshold values (for example, Qpthresh, Qpthresh1, Qpthresh2, and Qpcalthresh, described above) are selected. One exemplary decision tree is as follows:

If |Qp−Qpcal| is less than or equal to Qpthresh, then Q=Qa;

If |Qp−Qpcal| is greater than Qpthresh, then Q=skip reading.

Another version of a decision tree is as follows:

If Qp/Qpcal is greater than or equal to Qpthresh1 and Qp/Qpcal is less than or equal to Qpthresh2, then Q=Qa.

If Qp/Qpcal is less than Qpthresh1 or Qp/Qpcal is greater than Qpthresh2, then Q=skip reading.

The most important attribute is typically placed at the root of a decision tree. For example, in one embodiment of the present invention the root attribute is the current skin conductance value reading. In another embodiment, body temperature may be the root attribute. Alternatively, |Qp−Qpcal| or Qp/Qpcal could be used as the root attribute.

Further, thresholds need not be established a priori. An algorithm can learn from a database record of an individual subject's active collection reservoir glucose readings, passive collection reservoir glucose readings, body temperature, and skin conductance readings (as discussed herein). The algorithm can train itself to establish threshold values based on the data in the database record.

In addition, raw data obtained using the analyte monitoring device can be analyzed to develop sweat/temperature correction algorithms. For example, the raw data can be analyzed to include corrections based on such parameters as data from the skin conductivity probes, temperature readings, and the characteristics of both the anodic and cathodic biosensor signals (as discussed herein). This data can be taken into account by algorithms to provide correction and recalculation of glucose readings. Such algorithms can be included in firmware and/or software of the analyte monitoring device, for example, in one or more microprocessors programmed to control the analyte monitoring device and to execute such algorithms.

The success of a particular algorithm may be evaluated by statistical criteria to gauge the performance of the analyte monitoring device under the selected conditions (e.g., sweat and/or temperature changes). For example, a series of fingerstick blood glucose measurements (at least one per hour) can be used for comparison of values obtained from a glucose monitoring device, for example, a GlucoWatch G2 biographer. These blood values are matched in time with the GlucoWatch G2 biographer readings. Statistics used to evaluate performance include difference statistics between the GlucoWatch G2 biographer readings and the blood glucose values, regression analysis, Clark Error Grid analysis, and analysis of error and bias at different glucose levels. Usability, in terms of number and distribution of skipped readings, is also evaluated. The criteria for success of either of these correction techniques is a significant reduction in the number of readings skipped during periods of perspiration and/or temperature change while maintaining the accuracy of the readings.

Correction of the analyte readings, for example, glucose readings, may be accomplished using parameters collected during the measurement (e.g., the sweat probe (skin conductivity) measurement, temperature measurements, various parameters in the biosensor readings, including background, kinetic components of the biosensor signal, and parameters of the biosensor at the iontophoretic anode which is measuring mostly compounds other than glucose during non-sweating periods, but which would measure glucose as well which enters the gel during periods of sweating).

By selecting parameters and allowing an algorithm to train itself based on a database record of selected parameters for an individual subject or group of subjects, the algorithm can evaluate each parameter as independent or combined correction factors. Thus, the sweat/temperature model is being trained and the algorithm determines what parameters are the most important indicators.

Receiver Operating Characteristic (ROC) curve analysis is another threshold optimization means. It provides a way to determine the optimal true positive fraction, while minimizing the false positive fraction. A ROC analysis can be used to compare two classification schemes, and determine which scheme is a better overall predictor of the selected event (e.g., comparison of the parameter relationships described herein above in Section 2.2.3). ROC software packages typically include procedures for the following: correlated, continuously distributed as well as inherently categorical rating scale data; statistical comparison between two binormal ROC curves; maximum likelihood estimation of binormal ROC curves from set of continuous as well as categorical data; and analysis of statistical power for comparison of ROC curves. Commercial software for structuring and execution of ROC is available (e.g., Analyse-It for Microsoft Excel, Analyse-It Software, Ltd., Leeds LS 12 5XA, England, UK; MedCalc®, MedCalc Software, Mariakerke, Belgium; AccuROC, Accumetric Corporation, Montreal, Quebec, CA).

Related techniques that can be applied to the above analyses include, but are not limited to, Decision Graphs, Decision Rules (also called Rules Induction), Discriminant Analysis (including Stepwise Discriminant Analysis), Logistic Regression, Nearest Neighbor Classification, Neural Networks, and Naive Bayes Classifier.

One or more microprocessors of the present invention can be programmed to execute the decision trees, algorithms, techniques, and methods described herein above. Analyte monitoring devices of the present invention typically comprise such one or more microprocessors.

Experimental

The following examples are put forth so as to provide those of ordinary skill in the art with a complete disclosure and description of how to make and use the devices, methods, and formulae of the present invention, and are not intended to limit the scope of what the inventors regard as the invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

### Evaluation of Passive Gel as Sweat/Temperature Detection System

The following experiments investigated the feasibility of using the GlucoWatch G2 biographer with a passive sequence (no iontophoresis) in order to detect sweat. Two conditions were studied:
Condition 1: Control (sequence with iontophoresis)
Condition 2: Passive (sequence without iontophoresis) Six subjects participated in this study. Each subject wore eight research GlucoWatch G2 biographers (four per condition), two on the lower arms, four on the upper arms, and two on the chest. The GlucoWatch G2 biographers were applied in a left/right symmetrical fashion so that all active systems (condition 1) were applied to the left side of the body and all passive systems (condition 2) were applied to the right side of the body.

Two subjects exercised at the following elapsed times: 3:00 hours, 4:05 hours, and 5:10 hours. Two other subjects exercised at the following elapsed times: 3:20 hours, 4:25 hours, and 5:50 hours. The remaining two subjects exercised at the following elapsed times: 3:40 hours, 4:45 hours, and 5:50 hours.

Subjects exercised at 65% of their maximum heart rate or less. Each exercise session lasted thirteen minutes and the sessions were staggered to correspond with different parts of the iontophoresis extraction cycle of the GlucoWatch G2 biographer.

The study duration was 8 hours 18 minutes. Fingerprick samples were taken for glucose determination, two per hour (at 55 and 15 minute points) from ET 0:55 through one hour after last exercise session was completed. Subjects were fasting in order to obtain relatively constant blood glucose levels. The subjects' fasts began 90 minutes prior to the start of the study and continued until 45 minutes after the last exercise session ended. Reference blood measurements were taken twenty minutes prior to the corresponding GlucoWatch biographer measurement to account for the twenty minutes lag time of the GlucoWatch biographer glucose measurement.

An adjusted nC value was calculated by taking the nC value for a particular sensor at a particular time and subtracting a linear best fit of the nC signal for non-sweat affected readings versus elapsed time. Raw and adjusted nC tables were created by separating sweat and non-sweat events as determined by the skin conductivity sensors present in the GlucoWatch biographers. NanoCoulomb (nC) signals were reported for Sensor A and Sensor B, separately, as well as for the sum of Sensors A and B. Although active and passive systems were applied to the same position on opposite arms, a good correlation between the conditions was seen to exist. Assuming left/right symmetry, the passive systems (not extracting glucose via iontophoresis) were measuring a mix of everything that was electroactive in sweat, including glucose and interfering species. The actual amount of glucose in sweat may not be strongly correlated to the signal measured as sweat may contain both glucose and interfering species. However, large adjusted nC signals for the sweat affected cycles compared with those very near to zero for the non-sweat affected cycles were evidence that there was a substantial nC signal due to sweat.

A plot including data from all six subjects for active versus passive signal differences for sweat and non-sweat events is shown in FIG. 12. FIG. 15 and FIG. 16 graphically illustrate how the values in FIG. 12 were obtained. In FIG. 15, the plot is of nC signal at the cathode (Qa) for an active collection reservoir/sensing electrode (i.e., extraction with iontophoresis) on the y-axis, and elapsed time on the x-axis. The dots represent the nC signals, the line represents a best-fit linear regression of the nC data. The “x”s represent nC signals at time points associated with perspiration events. The double-headed arrows represent ΔnC=Qa−Qa(linear regression value at time A), wherein ΔnC=Qas+Qat. The differences, ΔnC=Qa−Qa(linear regression value at time A), were plotted in FIG. 12 as ΔnC Active on the y-axis. Qa(linear regression value at time A) is the best fit of the Qa signal when there is no sweating or temperature perturbation, which is the best estimate of Qag, with the linear fit accounting for signal decay in the Qag signal that normally occurs over time.

In FIG. 16, the plot is of nC signal at the cathode (Qp) for a passive collection reservoir/sensing electrode (i.e., no extraction with iontophoresis) on the y-axis, and elapsed time on the x-axis. The dots represent the nC signals, the line represents a best-fit linear regression of the nC data. The “x”s represent nC signals at time points associated with perspiration events. The double-headed arrows represent ΔnC=Qp−Qp(linear regression value at time A), wherein ΔnC=Qps+Qpt. These differences, ΔnC=Qp−Qp (linear regression value at time A), were plotted in FIG. 12 as ΔnC Passive on the x-axis. Qp (linear regression value at time A) was the best fit of the Qp signal when there was no sweating or temperature perturbation, which was the best estimate of Qpp, with the linear fit accounting for any signal decay in the Qpp signal that normally occurred over time.

In FIG. 12, the non-sweat events were more or less clumped near the origin of the graph because the corrected values were calculated by taking the difference from a best fit regression of non-sweat events only. This difference should be very close to zero. Conversely, adjusted values for the true positive sweat events should be seen more toward the upper right quadrant of the graph, due to the larger differences these nC values should have from the best fit regression. The value of this graph is found upon observation of the non-sweat events that were not closely clumped near the origin and sweat events that were clumped near the origin. These points represented false negatives and positives, respectively, that could be avoided with the implementation of an improved sweat detection system. The slope of the linear regression was substantially affected by two sweat-event outliers. Removal of these points resulted in a slope of 1.1024 and an intercept of 0.19 μC. The slope close to unity and the small y-intercept suggested that not only can the passive collection reservoir/sensing electrode be used for detection of sweat events, but for correction of data associated with sweat events as well.

A plot including data from all six subjects for adjusted active versus adjusted passive nC signals for sweat and non-sweat events is shown in FIG. 13. The difference between FIG. 12 and FIG. 13 is that latter uses passive signal values that were adjusted from the calibration value (signal at 2:15 ET); that is, FIG. 13 used (Qp−Qpcal) as the estimate of (Qps+Qpt). This was possible because there was minimal signal decay for the passive signal. FIG. 15 and FIG. 17 graphically illustrated how the values in FIG. 13 were obtained. FIG. 15 was described above. The differences, ΔnC=Qa−Qa (linear regression value at time A), were plotted in FIG. 13 as ΔnC Active on the y-axis.

In FIG. 17, the plot is of nC signal at the cathode (Qp) for a passive collection reservoir/sensing electrode (i.e., no extraction with iontophoresis) on the y-axis, and elapsed time on the x-axis. The line represented the nC signal at calibration (Qpcal). The “x”s represented nC signals at time points associated with perspiration events. The double-headed arrows represent ΔnC=Qp−Qpcal, wherein ΔnC=Qps+Qpt. These differences, ΔnC=Qp−Qpcal, were plotted in FIG. 13 as ΔnC Passive Adjusted from Cal on the x-axis.

In FIG. 13, the plot shows adjusted active (ΔnC=Qas+Qat) versus adjusted passive (ΔnC=Qpt+Qps≈Q−Qpcal). This data supported the use of a passive collection reservoir/sensing electrode in the GlucoWatch biographer to provide selectivity and/or compensation for sweat associated values. Use of a passive collection reservoir/sensing electrode allowed for the calculated nC signal for a particular time point to be analyzed with respect to the calibration value. Based on this information it can then be determined whether or not that point should be screened out or corrected. The slope of the linear regression performed on the data in FIG. 13 was affected by two sweat-event outliers. Removal of these points resulted in a slope of 0.981 and an intercept of 0.24 μC.

Results from this study supported the use of a passive collection reservoir/sensing electrode (i.e., collection of sample without application of an iontophoretic current followed by detection of analyte) not only for selective screening of data associated with sweat events, but for correction of data associated with sweat events as well. For example, data points at ETs A, B, and C, presented in FIG. 15 and FIG. 17, illustrate applications of several aspects of the present invention. At time point A, Q=Qa−Qp, that is, Qp is used as a correction for the contribution to input signal (Q) related to sweat (Qps) and temperature factors (Qpt), where Qp=Qps+Qpt. At time point B, the contribution of Qp to overall signal, during a period of sweating, is negligible. Accordingly, a data screen can be applied in this that allows Q=Qa without any further correction. This is an example of improved data selectivity, wherein even though a period of sweat has been noted the measured value is substantially unaffected by the sweat event. A different example of improved data selectivity is illustrated at time point C, wherein Qp≈Qa. In this situation a data screen may be applied to skip the measurement value at this time point due to an overwhelming contribution to signal by the sweat related signal.

Further, the following relationship involving a proportionality factor (k), discussed above in Section 2.2.3, can be deduced from the data presented in FIG. 13: Q=Qa−k(Qp−Qpcal). Based on the data in FIG. 13:
Q=Qa−(Qas+Qat), where (Qas+Qat)=k(Qp−Qpcal)
Q=Qa−k(Qp−Qpcal), where k=slope=(Qas+Qat)/(Qp−Qpcal).

In addition, a threshold may be set (e.g., Qpthresh) based on analysis of the data, wherein measurement values associated with a passive signal above a certain value are skipped. This threshold can then be used as a data screen. An example of one such Qpthresh is shown by the vertical dotted line in FIG. 18 (which corresponds to the data shown in FIG. 13).

A similar analysis can be applied to FIG. 15 and FIG. 16.

As is apparent to one of skill in the art, various modification and variations of the above embodiments can be made without departing from the spirit and scope of this invention. Such modifications and variations are within the scope of this invention.

What is claimed is:

1. An analyte monitoring device comprising,
(A) One or more collection reservoirs adapted for contact with a skin or mucosal surface of a subject, wherein (i) movement of said analyte into said collection reservoirs is enhanced by a transdermal or transmucosal sampling method, and (ii) during use of said device at least one collection device is placed in operative contact with an analyte sensing device; and
(B) One or more collection reservoirs adapted for contact with a skin or mucosal surface of a subject, wherein (i) movement of said analyte into said collection reservoirs not enhanced by said transdermal or transmucosal sampling method, and (ii) during use of said device at least one collection device is placed in operative contact with an analyte sensing device.

2. The analyte monitoring device of claim 1, wherein during use of said device at least one collection reservoir of (B) is in contact with a thermistor.

3. The analyte monitoring device of claim 1, wherein the physical characteristics of at least one collection reservoir of (A) are substantially the same as the physical characteristics of at least one collection reservoir of (B).

4. The analyte monitoring device of claim 3, wherein the at least one collection reservoir of (A) comprises a hydrogel.

5. The analyte monitoring device of claim 1, wherein said analyte sensing device is a device that detects analyte electrochemically.

6. The analyte monitoring device of claim 5, wherein said analyte sensing device comprises a sensing electrode.

7. The analyte monitoring device of claim 6, wherein the physical characteristics of the sensing electrode in contact with at least one collection reservoir of (A) has substantially the same physical characteristics of the sensing electrode in contact with at least one collection reservoir of (B).

8. The analyte monitoring device of claim 6, wherein said analyte sensing device further comprises an enzyme to facilitate electrochemical detection of the analyte.

9. The analyte monitoring device of claim 8, wherein said analyte is glucose and said enzyme comprises glucose oxidase.

10. The analyte monitoring device of claim 6, further comprising iontophoretic electrodes in contact with said one or more collection reservoirs of (A).

11. The analyte monitoring device of claim 1, wherein a collection reservoir of (B) comprises first and second surfaces, said first surface is in contact with a sensing device and said second surface is in contact with a membrane substantially impermeable to analyte, and said membrane is adapted for contact with said skin or mucosal surface.
